(19) United States
(12) Patent Application Publication
(10) Pub. No.: US 2026/0276418 A1
(43) Pub. Date: Sep. 17, 2026

(54) ADAPTER DEVICE FOR A SENSOR DEVICE, ADAPTER ARRANGEMENT, ADAPTER SYSTEM, MEASURING DEVICE HAVING ADAPTER DEVICE AND SENSOR DEVICE, AND METHOD

(71) Applicant: Bausch + Stroebel SE + Co. KG, Ilshofen (DE)

(72) Inventor: Robin Hofmann, Neuenstein (DE)

(21) Appl. No.: 19/673,506

(22) Filed: May 11, 2026

Related U.S. Application Data
(63) Continuation of application No. 18/455,306, filed on Aug. 24, 2023, now abandoned.
Application No. 18/455,306 is a continuation of application No. PCT/EP2022/054890, filed on Feb. 25, 2022.

(30) Foreign Application Priority Data
Feb. 25, 2021 (DE) 10 2021 104 558.4

(51) Int. Cl. G01F 1/667; G01F 1/66

(52) U.S. Cl. CPC G01F 1/667; G01F 1/662

(57) ABSTRACT

An adapter device, method, adapter arrangements, adapter system and measuring device. The adapter device can attach a sensor device having a first sensor part and a second sensor part to a pharmaceutical fluid conduit. The adapter device includes a first adapter part and a second adapter part. The first adapter part has a first sensor receptacle for the first sensor part and the second adapter part has a second sensor receptacle for the second sensor part. A fluid conduit receptacle is arranged between or formed by the adapter parts to receive the fluid conduit. The first and second sensor receptacles are arranged on the first and second adapter parts on sides facing away from the fluid conduit receptacle. Contact elements of the first sensor part and the second sensor part engage in or extend through through-openings for direct abutment against the fluid conduit in a planar manner.

### CROSS-REFERENCE TO RELATED APPLICATIONS

This application is a continuation of U.S. application Ser. No. 18/455,306, filed on Aug. 24, 2023, which is a continuation of International Application No. PCT/EP2022/054890, filed on Feb. 25, 2022, and claims priority to German Application No. 10 2021 104 558.4, filed on Feb. 25, 2021. The contents of U.S. application Ser. No. 18/455,306, International Application No. PCT/EP2022/054890 and German Application No. 10 2021 104 558.4 are incorporated herein by reference in their entireties and for all purposes.

### FIELD

The present disclosure relates to an adapter device for attaching a sensor device to a pharmaceutical fluid conduit, in particular pharmaceutical tubing, the sensor device having a first sensor part and a second sensor part.

In addition, the present disclosure relates to a method for attaching such a sensor device to the fluid conduit by means of an adapter device.

The present disclosure further relates to adapter arrangements having a first adapter device and at least one second adapter device.

The present disclosure also relates to an adapter system having a plurality of adapter devices.

In addition, the present disclosure relates to a measuring device having at least one sensor device and at least one adapter device.

The present disclosure relates to the use of a sensor device in a pharmaceutical plant for processing pharmaceutical containers. Such containers, for example vials, syringes, carpules and/or ampoules, are filled in the plant with a pharmaceutical substance (agent), in particular a liquid. It is known here to provide the desired quantity of the pharmaceutical substance by means of a metering unit. As an alternative or in addition to the use of a metering unit, it may be desirable to monitor, by means of a sensor device, the quantity of the substance supplied via the pharmaceutical fluid conduit.

Sensor devices for measuring the flow (volumetric flow rate) through a fluid conduit, which have a first sensor part and a second sensor part, are known. The sensor parts are arranged on opposite sides of the fluid conduit. Such sensor devices are offered, for example, by Keyence Deutschland GmbH (http://www.keyence.de) in the FD-X model series.

Conventional fastening devices are available for the sensor devices in order to attach the sensor parts to the fluid conduit. In practice, these fastening devices prove to be unsuitable for use in pharmaceutical plants, in particular if the fluid conduits are deformable tubing, for example made of a silicone material. However, the use of such tubing is desirable or even required in a pharmaceutical environment and for regulatory approval (e.g., by the FDA).

### BACKGROUND

An object underlying the present disclosure to provide an adapter device for a sensor device and a method for attaching a sensor device to a fluid conduit. In addition, an object of the present disclosure is to provide an adapter arrangement having two or more adapter devices for versatile use with different fluid conduits and a measuring device having an adapter device and a sensor device.

### SUMMARY

In a first aspect of the disclosure, an adapter device for attaching a sensor device having a first sensor part and a second sensor part to a pharmaceutical fluid conduit, in particular to pharmaceutical tubing that has a direction of extension is provided. The adapter device comprises a first adapter part and a second adapter part. The first adapter part comprises or forms a first sensor receptacle for the first sensor part and the second adapter part comprises or forms a second sensor receptacle for the second sensor part. The adapter parts are directly or indirectly interconnectable, and the adapter device comprises a fluid conduit receptacle which is arranged between the adapter parts or formed by the adapter parts and which is designed to receive the fluid conduit. The first sensor receptacle is arranged on the side of the first adapter part facing away from the fluid conduit receptacle and the second sensor receptacle is arranged on the side of the second adapter part facing away from the fluid conduit receptacle. A respective through-opening is formed on the first sensor receptacle and on the second sensor receptacle, and a respective contact element of the first sensor part and of the second sensor part engages in the respective through-opening or extends therethrough for direct abutment against the fluid conduit in a planar manner.

In a second aspect of the disclosure, a method for attaching a sensor device having a first sensor part and a second sensor part to a fluid conduit by means of an adapter device is provided. The method comprises:
connecting the first sensor part to the first adapter part;
connecting the second sensor part to the second adapter part;
arranging the fluid conduit in the fluid conduit receptacle; and
connecting the adapter parts to one another, the fluid conduit being arranged between the adapter parts in such a way that the contact elements of the sensor parts directly abut against the fluid conduit without intermediate elements.

In a third aspect of the disclosure, an adapter arrangement is provided. The adapter arrangement comprises a first adapter device in accordance with the first aspect and at least one second adapter device in accordance with the first aspect, wherein the fluid conduit receptacle of the first adapter device and the fluid conduit receptacle of the at least one second adapter device are of different sizes for receiving fluid conduits of different diameters.

In a fourth aspect of the disclosure, an adapter arrangement is provided. The adapter arrangement comprises an adapter device in accordance with the first aspect comprising a first intermediate part. The adapter arrangement further comprises at least one second intermediate part. The fluid conduit receptacles of the first intermediate part and the at least one second intermediate part are of different sizes for receiving fluid conduits of different diameters.

In a fifth aspect of the disclosure, an adapter arrangement is provided, in particular in accordance with the fourth aspect. The adapter arrangement comprises an adapter device in accordance with the first aspect comprising a first receiving part. The adapter arrangement further comprises at least one second receiving part, wherein the receiving parts each comprise at least one adapter part receptacle for one of the adapter parts. An offset in the extension direction of the sensor receptacles of the adapter parts connected to the receiving parts is different from one another and in particular is the greater, the larger the diameter of the fluid conduit to be used with the respective adapter device.

In a sixth aspect of the disclosure, an adapter system is provided. The adapter system comprises a plurality of adapter devices in accordance with the first aspect having a respective first adapter part and a respective second adapter part and/or at least one adapter arrangement in accordance with the third, fourth and/or fifth aspect, wherein preferably the first adapter parts and/or the second adapter parts are interconnected or are interconnectable.

In a seventh aspect of the disclosure, a measuring device comprising at least one adapter device in accordance with the first aspect and/or at least one adapter arrangement in accordance with the third, fourth and/or fifth aspect and/or at least one adapter system in accordance with the sixth aspect is provided. At least one sensor device has a first sensor part and a second sensor part, wherein the first sensor part is connected to the first adapter part and the second sensor part is connected to the second adapter part of the respective adapter device. Preferably, at least one fluid conduit is arranged in the fluid conduit receptacle between the adapter parts. The adapter parts are directly or indirectly interconnected, and the contact elements of the sensor parts directly abut against the at least one fluid conduit without intermediate elements.

### BRIEF DESCRIPTION OF THE DRAWINGS

The foregoing summary and the following description may be better understood in conjunction with the drawing figures, of which:

FIG. 1 shows a perspective representation of a measuring device in accordance with the disclosure, comprising an adapter device in accordance with the disclosure, a holding part also being shown;

FIG. 2 shows another perspective representation of the measuring device from FIG. 1;

FIG. 3 shows an exploded representation of the measuring device with a holding part from FIG. 1;

FIGS. 4 and 5 show perspective representations of a first adapter part of the adapter device from FIG. 1;

FIGS. 6 and 7 show perspective representations of a second adapter part of the adapter device from FIG. 1;

FIG. 8 shows a sectional view along the line 8-8 in FIG. 1;

FIG. 9 shows a sectional view along the line 9-9 in FIG. 1;

FIG. 10 shows a sectional view along the line 10-10 in FIG. 8;

FIG. 11 shows a sectional view along the line 11-11 in FIG. 8;

FIG. 12 shows a schematic representation of an adapter arrangement in accordance with the disclosure having three adapter devices;

FIG. 13 shows a perspective representation of another measuring device in accordance with the disclosure, comprising an adapter device in accordance with the disclosure and a sensor device;

FIG. 14 shows an exploded representation of the measuring device from FIG. 13;

FIG. 15 shows a sectional view along the line 15-15 in FIG. 13;

FIG. 16 shows a sectional view along the line 16-16 in FIG. 15;

FIG. 17 shows a sectional view along the line 17-17 in FIG. 15;

FIG. 18 shows a schematic representation of an adapter arrangement in accordance with the disclosure;

FIG. 19 shows a perspective representation of another measuring device in accordance with the disclosure, comprising an adapter device in accordance with the disclosure and a sensor device;

FIG. 20 shows an exploded representation of the measuring device from FIG. 19;

FIG. 21 shows a sectional view along the line 21-21 in FIG. 19;

FIG. 22 shows a representation of a receiving part of the adapter device from FIG. 19, the representation showing two plan views of the receiving part from opposing sides;

FIG. 23 shows a schematic representation of an adapter arrangement in accordance with the disclosure;

FIG. 24 shows a schematic representation showing a fluid conduit receptacle of an adapter device in accordance with the disclosure and tubing arranged in the fluid conduit receptacle in the direction of extension thereof,

FIG. 25 shows a perspective partial representation of a measuring device in accordance with the disclosure having an adapter system in accordance with the disclosure, comprising a plurality of adapter devices and a plurality of sensor devices, without fluid conduits being shown;

FIG. 26 shows an enlarged partial representation of the measuring device from FIG. 25, partially in an exploded representation;

FIGS. 27 to 30 show schematic representations of other preferred embodiments of the adapter system in accordance with the disclosure;

FIG. 31 shows a perspective representation of another measuring device in accordance with the disclosure, comprising an adapter device in accordance with the disclosure and a sensor device;

FIG. 32 shows a perspective representation of the measuring device from FIG. 31 in an exploded representation;

FIG. 33 shows a perspective representation of an intermediate part of the adapter device;

FIG. 34 shows another perspective representation of the adapter device without the intermediate part being shown;

FIG. 35 shows a sectional view along the line 35-35 in FIG. 31, a holding part for the adapter parts also being shown when used in an adapter system in accordance with FIGS. 37 and 38;

FIG. 36 shows a schematic representation of another adapter arrangement in accordance with the disclosure;

FIG. 37 shows a perspective partial representation of another measuring device in accordance with the disclosure having an adapter system in accordance with the disclosure, comprising a plurality of adapter devices and a plurality of sensor devices, with an adapter assuming a maintenance state; and

FIG. 38 shows a representation corresponding to FIG. 37, with the adapter part assuming a connected state with another adapter part.

### DETAILED DESCRIPTION

Although the disclosure is illustrated and described herein with reference to specific embodiments, the disclosure is not intended to be limited to the details shown. Rather, various modifications may be made in the details within the scope and range of equivalents without departing from the disclosure.

The present disclosure relates to an adapter device for attaching a sensor device having a first sensor part and a second sensor part to a pharmaceutical fluid conduit, in particular to pharmaceutical tubing, that has a direction of extension, the adapter device comprising a first adapter part and a second adapter part, the first adapter part comprising or forming a first sensor receptacle for the first sensor part and the second adapter part comprising or forming a second sensor receptacle for the second sensor part, the adapter parts being directly or indirectly interconnectable, and also a fluid conduit receptacle which is arranged between the adapter parts or is formed by the adapter parts and which is designed to receive the fluid conduit, the first sensor receptacle being arranged on the side of the first adapter part facing away from the fluid conduit receptacle and the second sensor receptacle being arranged on the side of the second adapter part facing away from the fluid conduit receptacle, a respective through-opening being formed on the first sensor receptacle and on the second sensor receptacle, and a respective contact element of the first sensor part and of the second sensor part engaging in the respective through-opening or extending therethrough for direct abutment against the fluid conduit in a planar manner.

The adapter device in accordance with the disclosure comprises a first and a second adapter part, which are assigned to the first and second sensor part, respectively. In the intended use of the adapter device, in which the sensor parts are arranged on the adapter parts and are preferably connected thereto, the first sensor part is arranged in the first sensor receptacle of the first adapter part and the second sensor part is arranged in the second sensor receptacle of the second adapter part. Signal-effective contact elements can extend through the respective through-opening or engage in the through-opening. In this way, the contact elements project from the respective sensor receptacle in the direction of the fluid conduit arranged between the adapter parts. The contact element can engage in the fluid conduit receptacle via the through-opening. This, in particular, offers the possibility of placing the contact elements against the fluid conduit in a planar and direct manner, without the arrangement of intermediate elements, preferably without gaps. It has been found in practice that, when a defined relative position of the adapter parts is ensured in the connected state, a temporally constant or substantially constant pressing force of the contact elements can be exerted on the fluid conduit. Furthermore, it has been found in practice that this is advantageous in particular in the case of tubing with a view to precise measurement of the volumetric flow rate. The contact elements preferably abut against the fluid conduit opposite one another.

The through-opening, into which the contact element engages, transitions, for example, into the fluid conduit receptacle so that the contact element can abut against the fluid conduit.

The fluid conduit receptacle, which is arranged on the side of the adapter part facing away from the sensor receptacle, can be delimited, for example, by an edge of the through-opening in the region of the through-opening.

In contrast to the disclosure, intermediate elements made of a deformable rubber material are used in the conventional fastening devices for the sensors of the FD-X model series of Keyence Deutschland GmbH. These intermediate elements have the disadvantage that they are arranged between the contact elements and the fluid conduit and deform during assembly and over time throughout the service life. The deformation also depends on how the fastening device is attached to the fluid conduit, which is disadvantageous for calibration. The deformation proves to be even more disadvantageous in the case of flexible tubing than in the case of rigid fluid conduits so that use of conventional fastening devices in a pharmaceutical environment does not lead to the desired sufficient accuracy of the flow measurement. Likewise, the provision of separate intermediate elements and thus additional components in the pharmaceutical environment is disadvantageous, if not with regard to approval, then at least with regard to cleaning.

The above disadvantages can be avoided by using the adapter device in accordance with the disclosure, via which the contact elements can abut directly without an intermediate element in the fluid conduit.

It is understood that the adapter device is preferably adapted to the dimensions of the fluid conduit, in particular the diameter thereof. In a preferred embodiment of the disclosure, at least one adapter arrangement is provided which is suitable for use with fluid conduits of different diameters in order to be able to adapt to different types of fluid conduits. This is discussed below.

In the intended connected state, the adapter parts receive the fluid conduit in the fluid conduit receptacle directly or indirectly between them. The sensor parts are arranged on the sides of the adapter parts facing away from the fluid conduit. It is further understood that, during the intended use, the fluid conduit is positioned in the fluid conduit receptacle and the relative position with respect to the adapter device is defined thereby.

The shape and/or size of the through-opening can be adapted to the contact element. In the embodiments explained here, for example, a substantially slit-shaped through-opening for a substantially strip-shaped contact element is used. In practice, it can prove advantageous, for example, to select the through-opening so as to be relatively small, for example in the case of ultrasonic sensors. Any stray fields of the ultrasound can, for example, be shielded by a base of the sensor receptacle and the measurement result can thereby be improved.

The respective sensor receptacle preferably comprises an abutment region for the sensor part, the respective through-opening being formed in the abutment region. A defined relative position of the sensor part with respect to the fluid conduit can be ensured at the abutment region. The abutment region preferably comprises a planar abutment element. The abutment region is arranged on the adapter part in particular on the side facing away from the fluid conduit receptacle.

It may be provided that the adapter device comprises a sealing element which is arranged between the sensor part and the abutment region. Each sensor part can be assigned its own sealing element. The sealing element comprises or forms, in particular, a cutout through which the contact element extends. The sealing element can be used, for example, to protect the sensor part in order to prevent the ingress of liquid escaping from the fluid conduit, for example. The sealing element is arranged, for example, between the sensor part and a base wall of the sensor receptacle and may be aligned with the through-opening on the adapter part.

It may be favorable if the respective sensor receptacle has a depression into which the sensor part can preferably be inserted in a positive-locking manner. This allows for a defined position of the sensor part on the adapter part. For example, the sensor part engages in a positive-locking manner in the depression enclosed by an edge.

The first sensor receptacle and the second sensor receptacle are advantageously configured identically.

It is advantageous if the respective adapter part can be rigidly connected to the sensor part assigned thereto. For example, the sensor part can be connected to the adapter part via at least one connecting element, for example a screw element. In a preferred embodiment, it may be provided that connecting elements engage from the side of the fluid conduit receptacle through the adapter part in the sensor receptacle in order to secure the sensor part on the adapter part.

The through-openings on the adapter parts advantageously have an extension in the direction of extension of the fluid conduit.

The through-openings may, for example, be slit-shaped.

Advantageously, the fluid conduit receptacle is designed such that the fluid conduit can be received therein in a positive-locking manner.

The fluid conduit receptacle is advantageously configured to extend in a straight line and/or is oriented in the direction of extension.

It proves advantageous if the fluid conduit receptacle comprises, for the fluid conduit, at least one abutment region that is preferably directly adjacent to the through-openings in the direction of extension. A defined position of the fluid conduit in the fluid conduit receptacle can be achieved at the abutment region, which is preferably adapted to a shape and/or a diameter of the fluid conduit.

Two abutment regions are preferably provided, on sides of the through-openings opposite one another in the direction of extension. This makes it possible, for example, to ensure a defined position of the fluid conduit on the adapter device on both sides of the through-openings so that a defined contact of the contact element with the fluid conduit can be ensured in the region of the through-opening.

It can be provided that each adapter part has at least one and preferably two abutment regions. Alternatively, it can be provided that only one of the adapter parts has at least one and preferably two abutment regions.

The at least one abutment region has, for example, a rounded portion in a sectional view transverse and, in particular, perpendicular to the direction of extension and is adapted to the contour of the fluid conduit. For example, the abutment region has the shape of a circular arc or is elliptical.

It is advantageous for the fluid conduit receptacle to have a concave recess that is adapted to the fluid conduit.

It is advantageous if the fluid conduit receptacle comprises at least one groove into which the fluid conduit can be inserted at least in part. In particular, the groove may comprise or form the above-mentioned at least one abutment region.

In a preferred embodiment, it may be provided that the fluid conduit is received completely in the at least one groove.

In a preferred embodiment of the disclosure, it is possible to provide two complementary grooves which are opposite one another transversely and, in particular, perpendicularly to the direction of extension and in which the fluid conduit engages in each case in part, for example in each case halfway. For example, in a connection direction of the adapter parts, the fluid conduit engages in each case in part in one of the grooves, so to speak at the top and at the bottom when the connection direction is viewed as being oriented in the height direction.

In the present case, a “groove” can in particular be regarded as a “channel,” and/or a recess which is configured at least in portions as a “channel.”

The cross section of the at least one groove is, for example, arc-shaped and, in particular, adapted to the shape and/or the diameter of the fluid conduit. For example, a circular arc shape or an elliptical shape of the at least one groove can be provided.

The cross section of the at least one groove is, for example, rectangular and, in particular, adapted to the shape and/or the diameter of the fluid conduit.

In particular in combination with an elliptical cross section, it can prove advantageous if a clear width of the at least one groove is smaller transversely and, in particular, perpendicularly to the direction of extension in a connection direction of the adapter parts than perpendicularly to the connection direction. In practice, it has been found that, for example, tubing can have dimensional tolerances. In the present embodiment, it is possible to place the contact elements against the tubing with a defined pressing force. Tubing of a larger diameter can be deformed somewhat in a width direction in which the clear width is greater than in the connection direction.

It may be advantageous if at least one projection is arranged or formed on one of the adapter parts in the direction of extension and projects beyond the other adapter part, in which projection a groove is formed into which the fluid conduit can be inserted at least in part and preferably completely. This makes it possible to extend the fluid conduit receptacle beyond the adapter part that does not have the projection. In this case, the fluid conduit is preferably received completely in the groove. By means of the at least one projection, a tubing is better protected from bending at the outlet between the adapter parts, for example.

The at least one projection is arranged or formed, for example, on the second adapter part.

It is advantageous if the adapter part has projections on two sides facing away from one another in the direction of extension. In this way, the aforementioned advantages can be achieved on both sides of the adapter device.

In the connected state, the other adapter part is preferably arranged in a positive-locking manner between the two projections in the direction of extension.

In a preferred embodiment of the disclosure, the fluid conduit receptacle is formed by the adapter parts.

For example, at least one groove extending in the direction of extension for the fluid conduit is arranged or formed on the first adapter part and/or on the second adapter part. For example, each adapter part comprises two grooves which are preferably directly adjacent to the respective through-opening and are opposite one another with respect to the through-opening. The through-opening can alternatively be regarded as a portion of the groove in which a base of the groove is perforated. In this respect, each adapter part may comprise, for example, a continuous groove.

In a preferred embodiment of the disclosure, the adapter device comprises an intermediate part which is formed separately from the adapter parts and is arranged between the adapter parts and comprises or forms the fluid conduit receptacle. The use of intermediate parts can be advantageous, for example, to forming an adapter arrangement, it being possible to use intermediate parts with fluid conduit receptacles of different sizes in order to adapt to different fluid conduit diameters.

The intermediate part can preferably be joined to the adapter parts without a joining gap.

In the context of the present application, “without a joining gap” can be understood to mean, for example, that components of the adapter device can be joined to one another so as to precisely fit together in at least one spatial direction and there are no steps in a spatial direction oriented at an angle thereto, taking into account any dimensional tolerances during the manufacture of the components.

It can be provided, for example, that a receptacle in which the intermediate part is received at least in part is formed for the intermediate part on at least one adapter part. Preferably, such a receptacle is formed on both adapter parts, the intermediate part engaging in each receptacle in part, preferably in each case halfway.

The receptacle is, for example, a depression, which is enclosed laterally by edges on the adapter part. The intermediate part arranged in the depression can be covered by the other adapter part. The adapter parts can abut against one another via the edges.

In a preferred embodiment of the disclosure, it may be provided that at least one receptacle for at least one of the adapter parts is provided on the intermediate part, into which receptacle the adapter part engages at least in portions. For example, a receptacle is assigned to each adapter part. In particular, a positive-locking fit of the adapter part in the receptacle is advantageous here. For example, it is advantageous if the adapter part abuts against edges of the receptacle in the direction of extension and/or a transverse direction oriented transversely and, in particular, perpendicularly thereto and preferably assumes a defined position in the receptacle.

In relation to the direction of extension, the intermediate part may, for example, be stepped with a centrally arranged receptacle, laterally next to which raised portions are present. The adapter part is preferably formed so as to be complementary to the intermediate part and may centrally comprise a projecting portion, laterally next to which portions recessed in relation to the direction of extension are present.

The intermediate part extends, for example, in the direction of extension over the entire length of at least one adapter part.

For example, the intermediate part is aligned with at least one adapter part at the end side in the direction of extension.

In one embodiment of the disclosure, it may be provided that the intermediate part projects beyond at least one adapter part on at least one side in the direction of extension.

The intermediate part may, for example, be substantially plate-shaped.

It may be provided that the intermediate part is arranged between the adapter parts in such a way that it abuts against the first adapter part and the second adapter part via corresponding abutment elements, said adapter parts not abutting against one another.

In a manner of speaking, the intermediate part may be arranged in a sandwich-like manner between the adapter parts and abut against the first adapter part and the second adapter part, which do not abut against one another. Corresponding abutment elements are preferably planar.

An outer contour of the intermediate part is aligned, for example, with an outer contour of the first adapter part and/or an outer contour of the second adapter part.

In a preferred embodiment of the disclosure, it is provided that the adapter device comprises a receiving part which comprises or forms at least one adapter part receptacle for at least one of the adapter parts, it being possible for the adapter part to be arranged preferably in a positive-locking manner in the adapter part receptacle. The adapter part can be arranged in a defined position in the adapter part receptacle. For example, this proves advantageous if the adapter part receptacle comprises or forms the fluid conduit receptacle. In this case, a defined relative position of the first and/or of the second sensor part with respect to the fluid conduit can be ensured.

The at least one adapter part receptacle is, for example, trough-shaped.

For example, in relation to the direction of extension, the receiving part has raised portions laterally next to the adapter part receptacle. For example, the adapter part can engage in the adapter part receptacle by means of a projecting portion.

Both adapter parts can be assigned a respective trough-shaped adapter part receptacle.

A positive-locking fit of a respective adapter part on the adapter part receptacle is advantageous, it being possible, for example, for the adapter part to abut against edges of the adapter part receptacle. Advantageously, a relative orientation of the adapter part and of the receiving part in the direction of extension and/or a transverse direction oriented transversely and, in particular, perpendicularly thereto takes place.

The respective other adapter part may be connectable, for example, to the first-mentioned adapter part and/or to the receiving part.

It is understood that, during the intended use, the at least one adapter part is arranged in the at least one adapter part receptacle and the other adapter part can be connected to this adapter part and/or connected to the receiving part.

The adapter part can, for example, be inserted into the adapter part receptacle in a connection direction of the adapter parts.

The at least one adapter part receptacle can preferably comprise wall arrangements that are spaced apart from one another and between which the at least one adapter part can be arranged. The wall arrangements are, for example, spaced apart from one another in the direction of extension and/or in a direction transverse and, in particular, perpendicular to the direction of extension.

A wall arrangement of the at least one adapter part receptacle, in particular the aforementioned wall arrangements, is rounded, for example, along an outer periphery of the adapter device. This improves the cleanability of the adapter device, for example.

It may be advantageous, in particular, if the receiving part comprises two adapter part receptacles, it being possible to arrange each adapter part in one of the receptacles at least in portions. This allows for a defined relative position of the adapter parts relative to one another and relative to the fluid conduit. The two adapter part receptacles are, in particular, arranged on sides of the receiving part facing away from one another and face a respective adapter part.

It may in particular be provided that the adapter part receptacles have an offset relative to one another in the direction of extension. As a result, for example, the sensor receptacles can have an offset relative to one another in the direction of extension. This may prove advantageous in certain types of sensor devices.

For sensor devices of the FD-X model series, sensor parts whose sensor signals are emitted at a measuring angle of substantially 45° relative to the direction of extension are used, for example. Due to the offset of the sensor receptacles from one another, the sensor parts also have an offset from one another in the direction of extension. This makes it possible to orient the sensor parts in such a way that the measurement signal (for example ultrasound) is ideally oriented in the direction of the other sensor part, with a view to accurate measurement of the volumetric flow rate.

In the last-mentioned embodiment in particular, the receiving part can, for example, be a so-called “format part,” which can be used together with otherwise format-independent adapter parts depending on the size, in particular the diameter, of the fluid conduit to be used. This is discussed below.

It is advantageous if the aforementioned intermediate part is, comprises or forms the receiving part. A preferred embodiment has the intermediate part with the fluid conduit receptacle for the fluid conduit, which intermediate part comprises or forms at least one adapter part receptacle and preferably two adapter part receptacles.

The at least one adapter part receptacle of the receiving part can, in particular, be the above-mentioned at least one receptacle of the intermediate part.

References to the receiving part can, in the present case, be regarded, in particular, as references to an intermediate part which comprises or forms at least one receptacle for at least one of the adapter parts. Receptacles of the intermediate part for the adapter parts can have an offset relative to one another in the direction of extension, as was explained above based on the example of the adapter part receptacles.

For example, the intermediate part projects in the direction of extension and/or in a direction transverse and, in particular, perpendicular to the direction of extension on at least one side of the adapter device beyond at least one of the adapter parts and comprises a wall arrangement of the at least one adapter part receptacle or forms such a wall arrangement.

The receiving part can, for example, have a plate-shaped portion which substantially comprises the fluid conduit receptacle and is positioned between the adapter parts. For example, wall arrangements of the respective adapter part receptacle can protrude from this portion.

It is advantageous if the adapter parts each comprise a preferably planar abutment element facing one another and abut against one another via the abutment elements in a connected state. This allows for a defined relative position of the adapter parts, which are advantageous for precise positioning of the contact elements on the fluid conduit.

The adapter parts are preferably joined to one another without a joining gap when they assume the connected state.

The adapter parts can advantageously be interconnected manually and/or without tools, as a result of which the adapter device can be handled in a simpler manner. This is important, in particular in a pharmaceutical environment.

The adapter parts can preferably be detachably interconnected. For example, the adapter parts are detached from one another in order to clean them or in order to attach them to a different fluid conduit.

Preferably, the adapter parts can be detached from one another manually and/or without tools. The advantages mentioned in connection with the connection process apply accordingly.

It may be favorable if the adapter parts comprise corresponding alignment elements for a defined relative position of the adapter parts in a connected state, the alignment elements being effective in the direction of extension and/or transversely and, in particular, perpendicularly to the direction of extension.

The alignment elements comprise, for example, at least one projection on an adapter part and at least one corresponding receptacle on the other adapter part.

It may be provided that the projection is configured as a strip and the receptacle is configured as a groove. In a preferred embodiment of the disclosure, the strip and the groove extend in parallel with the fluid conduit receptacle in the direction of extension.

Other configurations of the alignment elements are conceivable.

More than just one alignment element may be provided on a respective adapter part.

It may be provided that the sensor receptacles and thus the sensor parts are arranged or can be arranged on the adapter device at the same position in the direction of extension. The contact elements can also be arranged at the same position in the direction of extension.

In a preferred embodiment of the disclosure, the sensor receptacles have an offset relative to one another in the direction of extension. The advantage of such a configuration has already been discussed.

It is advantageous if the adapter parts can assume different relative positions and/or are displaceable relative to one another in the direction of extension. An adaptation of the adapter device to fluid conduits of different diameters can result in the distance between the sensor parts transverse and, in particular, perpendicular to the direction of extension (in particular in a connection direction of the adapter parts) being different. In order to orient the measurement signal toward the respective other sensor part in an ideal manner, it is advantageous if the sensor parts can be positioned differently relative to one another in the direction of extension. This can be implemented, for example, by providing different relative positions or by the possibility of displacement.

The adapter device is, for example, free from separate connecting elements for connecting the adapter parts to one another. In this way, the number of components of the adapter device can be kept low and the handling thereof can be improved. Fewer components further allow for easier cleaning of the adapter device, which is advantageous in particular when used in a pharmaceutical environment.

It has already been mentioned that the adapter parts can assume a connected state, the fluid conduit being held in the fluid conduit receptacle. Further provided is, in particular, a detached state in which the fluid conduit can be removed from the fluid conduit receptacle. The detached state can, in particular, be a maintenance state.

It is provided, for example, that the adapter device comprises a connecting part by means of which the adapter parts are interconnected directly or indirectly (for example via a holding part which is connected to an adapter part and to the connecting part) in the connected state and in the detached state and are preferably held against one another in a captive manner. After detaching and exposing the fluid conduit receptacle, the adapter parts still remain interconnected via the connecting part. This improves the handling of the adapter device.

The connecting part may, for example, be or comprise a flexible element. For example, the use of tubing is conceivable, an electrical connection line for at least one sensor part being guided through the tubing.

The adapter parts are preferably free from a positive-locking interconnection when they assume the connected state.

It is advantageous if the adapter parts are held against one another by magnetic force when they assume the connected state. The magnetic force is provided by means of magnetic elements, which are in particular permanent magnets. The use of NdFeB magnets is conceivable, for example.

It is accordingly advantageous if the adapter parts each comprise at least one magnetic element, which interact with one another in the connected state of the adapter parts.

The respective at least one magnetic element is, for example, cast, injected or pressed into the adapter part.

In a preferred embodiment of the disclosure, it is, for example, provided that a receptacle in which the magnetic element is arranged is formed on the adapter part for the respective at least one magnetic element. The receptacle is, for example, like a blind hole. For example, an opening of the receptacle is arranged on a side of the adapter part facing away from the respective other adapter part.

A cover element for closing the receptacle is advantageously provided. Closure without a joining gap is preferred in order to prevent dirt particles from settling.

After the magnetic element has been inserted into the receptacle, the cover element can, for example, be welded to the adapter part in order to prevent a gap as far as possible. For example, a plastics welding method can be used by means of which the cover element is preferably welded without gaps on the adapter part in order to close the receptacle.

A pressing element, for example a ring, can be provided, by means of which the magnetic element is held in a defined position in the adapter part.

The adapter part may comprise a cover element which covers two or more receptacles for magnetic elements and/or the sensor receptacle on the adapter part. In this way, separate cover elements can be avoided.

A plurality of magnetic elements is preferably arranged on each adapter part in order to allow for a reliable connection of the adapter parts to one another.

It can prove favorable if the magnetic elements are arranged along an imaginary peripheral line around the respective sensor receptacle. For example, in a plan view of the sensor receptacle, the magnetic elements are positioned at corner regions of an imaginary peripheral line surrounding the sensor receptacle.

In particular, four magnetic elements can be provided in each case.

For example, at least one magnetic element and/or a magnetizable element can be provided on the above-mentioned intermediate part and/or receiving part in order to allow for a connection to the adapter parts via magnetic force and/or to improve the connection thereof to one another via magnetic force. For example, a receptacle for the magnetic element and/or the magnetizable element may be provided on the intermediate part and/or receiving part. A cover element may be provided in order to close the receptacle. Closure without a joining gap or welding for preventing gaps may be provided. The magnetic element or magnetizable element is, for example, cast, injected or pressed into the receptacle.

In another embodiment, it may be provided that the intermediate part and/or the receiving part are free from magnetic elements, only the adapter parts comprising respective magnetic elements and a magnetic holding force acting through the intermediate part and/or the receiving part.

The adapter device is preferably configured to be suitable for pharmaceutical use. For this purpose, the materials of the adapter device have, for example, official approval (in particular FDA approval). For example, the adapter parts and, if applicable, the intermediate part are manufactured from materials suitable for pharmaceutical use, in particular with regulatory approval. Advantageous materials are, for example, stainless steel or POM (polyoxymethylene).

The adapter device is preferably designed such that the smallest possible number of gaps or corners is present in order to improve the cleaning properties. Undercuts and shadowing are preferably avoided.

The first adapter part and/or the second adapter part is preferably configured in one piece, with the exception, if present, of the magnetic elements and any cover elements as explained above.

The intermediate part, if present, is preferably configured in one piece.

The adapter device preferably has, on two sides facing away from one another in a transverse direction which is oriented transversely and, in particular, perpendicularly to the direction of extension and to the connection direction of the adapter parts, concave engagement regions which are delimited in the connection direction by the first adapter part and/or by the second adapter part. The user can reach into the engagement regions; they act as recessed grips, so to speak. This is advantageous, for example, in order to detach the adapter parts from one another, and improves the handling of the adapter device.

Preferably, mutually aligned lateral surfaces of the adapter parts are provided along at least one contiguous edge region of an outer periphery, in a plane transverse and, in particular, perpendicular to the connection direction of the adapter parts.

The adapter device may comprise a holding part on which an adapter part is secured or can be secured, it being possible to transfer the further adapter part from a connected state, in which it is connected directly or indirectly to the first-mentioned adapter part, into a maintenance state, in which the adapter part can be detachably secured to the holding part and the fluid conduit receptacle is exposed. The holding part advantageously provides a “parking position” for the detached adapter part when the maintenance state is assumed. This improves the handling of the adapter device. The adapter part to be detached and the holding part are advantageously interconnected via the above-mentioned connecting part. In a preferred embodiment, the detachable adapter part can be detachably secured to the holding part by means of magnetic force, for example.

It may be provided that the holding part is used to secure a plurality of adapter devices, for example as a component of an adapter arrangement in accordance with the disclosure or of an adapter system in accordance with the disclosure.

As mentioned at the outset, the present disclosure also relates to a method.

A method in accordance with the disclosure for attaching a sensor device having a first sensor part and a second sensor part to a fluid conduit by means of an adapter device of the type described above comprises the following steps, the first three of which are not necessarily to be carried out in this order:
connecting the first sensor part to the first adapter part;
connecting the second sensor part to the second adapter part;
arranging the fluid conduit in the fluid conduit receptacle; and
connecting the adapter parts to one another, the fluid conduit being arranged between the adapter parts in such a way that the contact elements of the sensor parts directly abut against the fluid conduit without intermediate elements.

In the method, it may be provided that an adapter device is used without an intermediate part. In this case, the fluid conduit can be positioned directly between the adapter parts. If an intermediate part is used, the fluid conduit can be positioned, for example, in the intermediate part and the latter has been or is positioned between the adapter parts.

The method in accordance with the disclosure has the advantages which have already been mentioned in connection with the explanation of the adapter device in accordance with the disclosure. Reference is made to the above statements. In particular, no intermediate elements are arranged between the contact elements of the sensor parts and the fluid conduit, against which the contact elements preferably abut without gaps.

Preferred exemplary embodiments of the method in accordance with the disclosure result from advantageous embodiments of the adapter device in accordance with the disclosure. In this regard, reference can be made to the above statements.

As mentioned at the outset, the present disclosure also relates to an adapter arrangement.

An adapter arrangement in accordance with the disclosure comprises a first adapter device of the type described above without an intermediate part and at least one second adapter device without an intermediate part, the fluid conduit receptacle of the first adapter device and the fluid conduit receptacles of the at least one second adapter device being of different sizes for receiving fluid conduits of different diameters. In this way, depending on which fluid conduit is used, the first or the at least one second adapter device can be used. The respective adapter device is adapted to the diameter of the fluid conduit to be used.

Furthermore, the first and the at least one second adapter device can be configured to be functionally identical.

The above-mentioned object is further achieved by an adapter arrangement in accordance with the disclosure, comprising an adapter device of the type described above, which comprises a first intermediate part and at least one second intermediate part, the fluid conduit receptacles of the first intermediate part and of the at least one second intermediate part being of different sizes for receiving fluid conduits of different diameters.

Depending on which fluid conduit is used, the intermediate part suitable for this purpose can be used together with the same adapter parts. It is understood that the intermediate parts can have identical dimensions, apart from the size of the fluid conduit receptacle. For example, the intermediate parts can also differ from one another in terms of their thickness depending on the dimensions of the fluid conduits. The intermediate part can be a so-called “format part.”

Such an adapter arrangement also offers the advantage, for example, that the sensor parts can remain connected to the adapter parts and only the intermediate part has to be replaced.

As already mentioned, the use with fluid conduits of different diameters can result in the distance between the sensor receptacles and thus between the sensor parts being varied. Accordingly, it is advantageous if the adapter parts can assume different relative positions in the direction of extension or are displaceable relative to one another, as already explained.

The above-mentioned object is further achieved by an adapter arrangement in accordance with the disclosure which comprises an adapter device of the type described above having a first receiving part and at least one second receiving part, the receiving parts each comprising at least one adapter part receptacle for one of the adapter parts, an offset in the direction of extension of the sensor receptacles of the adapter parts connected to the receiving parts being different from one another.

Preferably, two adapter part receptacles are provided.

Depending on which fluid conduit is used, the suitable receiving part can be used together with the same adapter parts. For this purpose, the fluid conduit receptacle is arranged, in particular, on the receiving parts or is comprised or formed by the receiving parts. For example, the larger the diameter of the fluid conduit to be used with the respective adapter device, the greater the offset. In this way, different relative positions of the sensor parts can be achieved in the direction of extension with a view to adjusting the measuring angle and to a precise measurement.

The distance between the adapter parts can be greater in this adapter arrangement, the larger the diameter of the fluid conduit.

The receiving parts are, in particular, so-called “format parts,” and the adapter parts can be format-independent. In this case, too, the sensor parts can, for example, remain connected to the adapter parts; only the receiving part is to be replaced.

The intermediate part may, in particular, be or form the receiving part, as already explained above.

As mentioned at the outset, the present disclosure also relates to an adapter system.

An adapter system in accordance with the disclosure comprises a plurality of adapter devices of the type described above having a respective first adapter part and a respective second adapter part and/or at least one adapter arrangement of the type described above.

The adapter system in accordance with the disclosure is preferably used with sensor devices, each adapter device being assigned a sensor device having a first sensor part and a second sensor part, as described above. In addition, the adapter device is in particular used with a respective fluid conduit, which can be assigned to the adapter device. The fluid conduits may be configured identically or differently. In particular, fluid conduits of different sizes, in particular different diameters, may be provided. The fluid conduits are, for example, tubing.

The adapter system can be used, for example, in a pharmaceutical plant in which a plurality of containers can preferably be filled at the same time by means of filling elements in a filling station, a so-called “multistation” filling station. An adapter device having a sensor device can be arranged on the fluid conduit to each filling element.

The adapter system in accordance with the disclosure is provided, in particular, for use with the measuring device in accordance with the disclosure or part of the measuring device.

It can be provided that the first adapter parts and/or the second adapter parts are interconnected or can be interconnected. For example, the handling of the adapter system can be facilitated when adapter parts are interconnected.

For connection, the adapter parts can be integrally formed, for example.

For example, a one-piece connection of the first adapter parts and/or of the second adapter parts to one another is conceivable.

In a preferred embodiment of the disclosure, the first adapter parts and/or the second adapter parts are formed separately from one another.

A preferred embodiment of the disclosure provides, for example, that in particular the second adapter parts are interconnected, the first adapter parts being formed separately from one another. The first adapter parts can preferably each be connected individually to the respective second adapter part. For example, the respective fluid conduit can thereby be separately inserted into the adapter device or detached therefrom.

It can be provided that the adapter devices are interconnected, for example via a holding part.

It is preferably provided that the respective first adapter parts and/or the respective second adapter parts are configured identically. As a result, the design of the adapter system is simplified just as handling for the user is facilitated.

The adapter devices are, for example, arranged or can be arranged laterally next to one another in a row. For example, the adapter devices are arranged laterally next to one another in a transverse direction which is oriented transversely and, in particular, perpendicularly to the direction of extension. The fluid conduits are preferably arranged in parallel with one another and, accordingly, so are the fluid conduit receptacles.

It has already been discussed that the adapter device and the adapter arrangement can comprise, for example, an intermediate part having the fluid conduit receptacle.

In a preferred embodiment of the adapter system, it is advantageous if the respective adapter device comprises at least one intermediate part. Reference is made to the above statements.

It may in particular be provided that a plurality of intermediate parts are assigned to a respective adapter device, said intermediate parts differing from one another with regard to the size of the respective fluid conduit receptacle. Accordingly, the adapter system can comprise adapter arrangements as described above.

The adapter parts are preferably format-independent, and the respective intermediate part is preferably a so-called “format part” which is adapted to the fluid conduit.

As already mentioned, the adapter device can comprise a receiving part which is formed, for example, by the intermediate part or is comprised thereby.

It is advantageous if the respective adapter device of the adapter system comprises at least one receiving part. Reference is made to the above statements in this regard. The at least one receiving part can comprise at least one adapter part receptacle.

The adapter system can comprise an adapter arrangement, a plurality of receiving parts being provided. The receiving parts can differ in that the offset of the sensor receptacles in the direction of extension is different, in particular depending on the size and especially on a diameter of the fluid conduit to be used.

The adapter parts may preferably be format-independent, and the receiving parts may be so-called “format parts” which can be used depending on the fluid conduit.

As mentioned at the outset, the present disclosure also relates to a measuring device.

A measuring device in accordance with the disclosure comprises at least one adapter device, at least one adapter arrangement and/or at least one adapter system of the type described above, at least one sensor device having a first sensor part and a second sensor part, the first sensor part being connected to the first adapter part and the second sensor part being connected to the second adapter part of the respective adapter device, and preferably at least one fluid conduit which is arranged in the fluid conduit receptacle between the adapter parts, the adapter parts being directly or indirectly interconnected and the contact elements of the sensor parts directly abutting against the at least one fluid conduit without intermediate elements, preferably without gaps.

The advantages that were already mentioned in connection with the explanation of the adapter device in accordance with the disclosure can also be achieved in the case of the adapter arrangements, the adapter system and the measuring device.

Advantageous embodiments of the adapter arrangements in accordance with the disclosure, of the adapter system in accordance with the disclosure and/or of the measuring device in accordance with the disclosure result from advantageous embodiments of the adapter device in accordance with the disclosure. Reference is made to the above statements.

Preferred embodiments of the adapter device in accordance with the disclosure, of the measuring device in accordance with the disclosure and of adapter arrangements in accordance with the disclosure are discussed below. Preferred exemplary embodiments of the method in accordance with the disclosure can be carried out using the adapter devices.

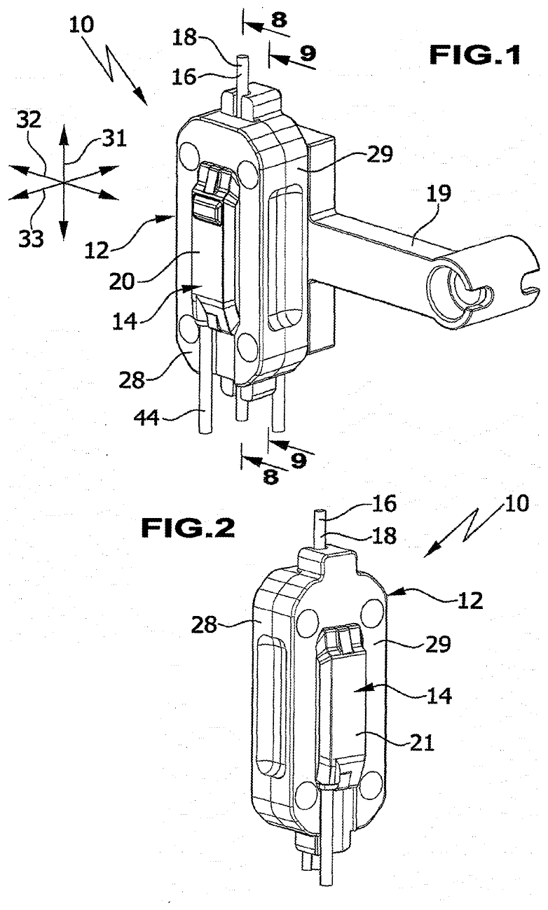

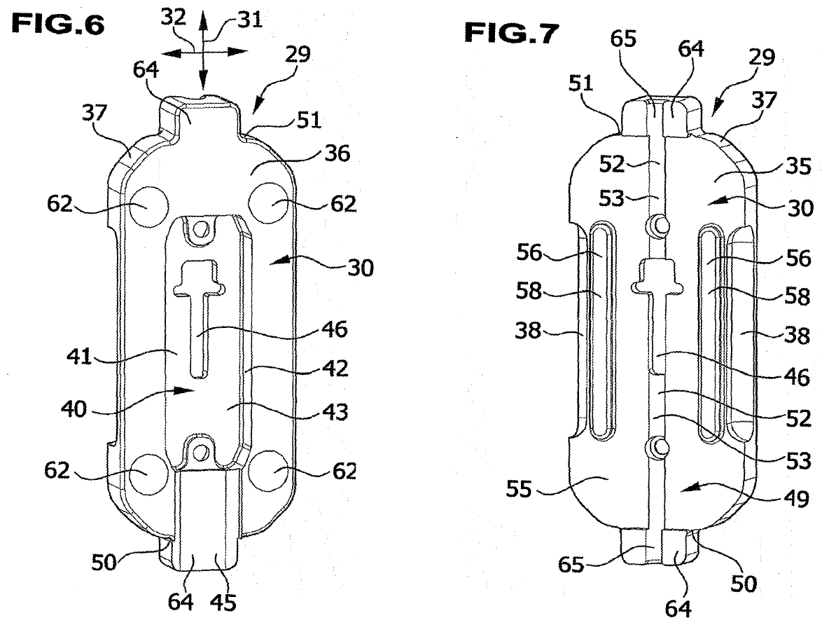

Identical reference numerals are used for the same and functionally equivalent features and components. Firstly, with reference to FIGS. 1 to 12, first preferred embodiments of the measuring device, or the adapter device and of the adapter arrangement are discussed. Subsequently, other preferred embodiments of the disclosure are presented, with only the most important differences being explained. Otherwise, the statements apply mutatis mutandis.

FIGS. 1 to 3 show a preferred embodiment of the measuring device in accordance with the disclosure, designated as a whole by reference numeral 10. The measuring device 10 comprises a first preferred embodiment of the adapter device in accordance with the disclosure, which is designated by reference numeral 12, a sensor device 14 and a fluid conduit 16, which is configured, in particular, as tubing 18. Reference numeral 19 denotes an optional holding part of or for the measuring device 10.

The measuring device 10 is provided and configured, in particular, for use in a pharmaceutical plant for processing pharmaceutical containers. The measuring device 10 is used, in particular, to measure the flow (volumetric flow rate) of a pharmaceutical substance to be filled into the containers, through the tubing 18 by means of the sensor device 14. The measuring device 10 can be used in addition or as an alternative to a metering unit of the pharmaceutical plant.

In the present example, the sensor device 14 is of the conventional type and is known per se. The adapter device 12 is used to attach the sensor device 14 to the tubing 18 in such a way that a measurement result that is as precise as possible can be achieved when determining the volumetric flow rate.

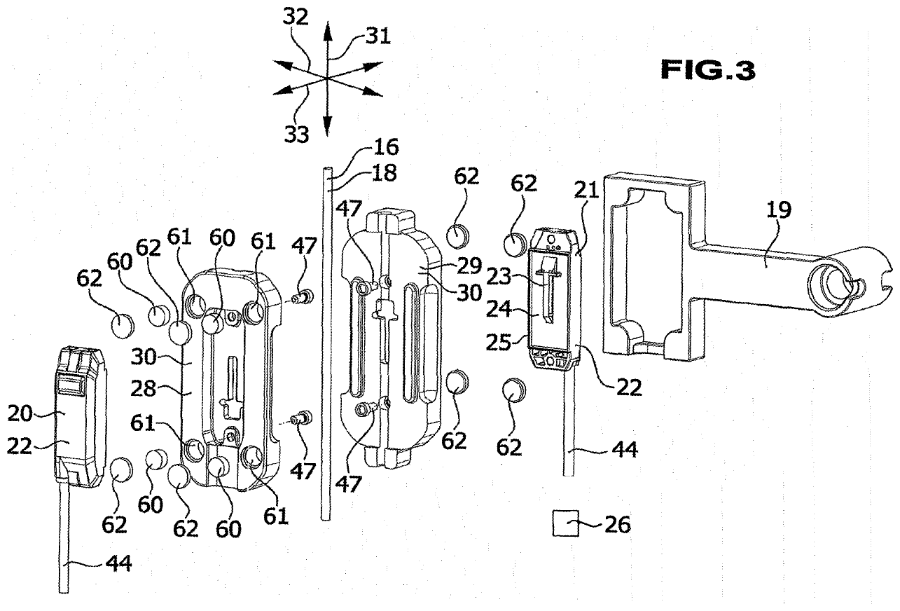

The sensor device 14 comprises a first sensor part 20 and a second sensor part 21. A respective sensor part 20, 21 forms a so-called “sensor head” with a housing 22. A signal-effective contact element 23 is arranged on the housing 22. In the present case, the contact element 23 is substantially strip-shaped, approximately having a T shape in plan view (FIG. 3).

The contact element 23 protrudes from a base surface 24 of the housing 22. The housing 22 further forms a projecting edge 25 enclosing the base surface 24 and the contact element 23.

In the intended use of the sensor device 14, the sensor parts 20, 21 are to be attached to opposing sides of the tubing 18, the contact elements 23 being opposite one another.

In the present example, the sensor device 14 is an ultrasonic sensor device. Each sensor part 20, 21 serves simultaneously as a transmitter and as a receiver, the transmission of ultrasound through the tubing 18 being determined. The speed of the liquid flowing through the tubing 18 and thus the volumetric flow rate can be determined depending on the sensor signal of the sensor means 14.

It is understood that the sensor device 14 and/or the measuring device in accordance with the disclosure can comprise an evaluation device 26 or can be operatively connected to an evaluation device 26. The evaluation device 26 is shown schematically in FIGS. 3, 14, 20 and 25. It may, for example, be a component of a control device of the pharmaceutical plant or be coupled to the control device.

The sensor device 14 is, for example, from the FD-X model series of Keyence Deutschland GmbH.

The adapter device 12 comprises a first adapter part 28 and a second adapter part 29.

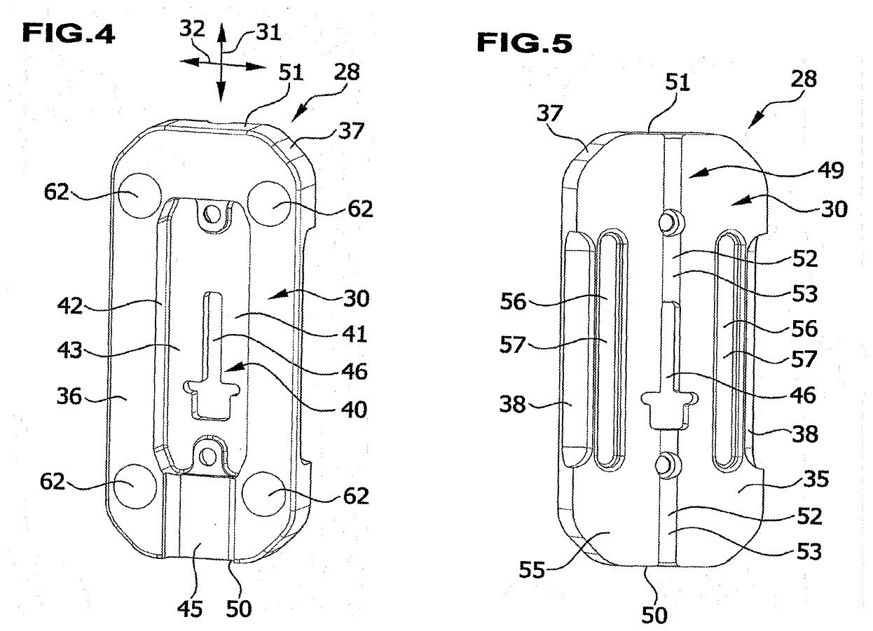

As is clear in particular from FIGS. 4 to 7, the adapter parts 28, 29 are configured to be functionally identical. Components of the adapter parts 28, 29 are partially identical and complementary to components of the respective other adapter part 29, 28.

Identical reference numerals are used for functionally identical components of the adapter parts 28, 29. Firstly, the adapter part 28 is discussed. The explanations in this regard also apply to the adapter part 29 so that repetitions can be avoided. Only the main differences between the adapter parts 28, 29 are described.

As can be seen in particular from FIGS. 4 and 5, the adapter part 28 has a base body 30. The base body 30 has an approximately cuboidal contour in a rough approximation. The base body 30 is approximately the size of the palm of a hand in order to facilitate handling of the adapter device 12 for a user.

The base body 30 is approximately disk-shaped or plate-shaped. In this case, the base body 30 has an extension in a direction of extension 31 that is defined by the tubing 18. An intended use of the adapter device 12 with the tubing 18 is assumed here.

A transverse direction 32 of the adapter part 28 is oriented transversely and, in particular, perpendicularly to the direction of extension 31. A third direction of the adapter part 28 is oriented transversely and, in particular, perpendicularly to the directions 31, 32. This third direction extends in a connection direction 33 for interconnecting the adapter parts 28, 29, as is explained below. The double arrow 33 in the drawing denotes the connection direction and the detachment direction.

The extension of the base body 30 in the direction of extension 31 is significantly greater than in the transverse direction 32. The extension in the transverse direction 32 is in turn significantly greater than in the connection direction 33.

The adapter part 28 has a first side 35 which, in the intended use, corresponding to the connected state of the adapter parts 28, 29, faces the adapter part 29. The first side 35 faces the tubing 18.

The adapter part 28 comprises a second side 36 which faces away from the adapter part 29. The second side 36 faces away from the tubing 18 and the fluid conduit receptacle mentioned below and, in the connected state of the adapter parts 28, 29, is an outer side, for example.

The sides 35, 36 are interconnected via a circumferential outer surface 37, which is a lateral surface in a first approximation. In the transverse direction 32, two recesses 38 are formed on the outer surface 37 so as to be spaced apart from one another.

On the second side 36, the adapter part 28 comprises a sensor receptacle 40 for receiving the sensor part 20. The sensor receptacle 40 is in the form of a depression 41, which is enclosed by a circumferential edge 42. The base of the depression 41 forms an abutment region 43 for the sensor part 20, in particular for the edge 25.

The sensor receptacle 40 is dimensioned such that the sensor part 20 can engage in a positive-locking manner in the sensor receptacle 40. In the present case, the abutment region 43 is planar.

A connection line 44 of the sensor part 20 can be guided through a recess 45 formed on the first side 35. The recess 45 adjoins the sensor receptacle 40.

A through-opening 46 is formed on the sensor receptacle 40. In the present case, the through-opening 46 is slit-shaped and has an extension in the direction of extension 31.

The through-opening 46 is arranged at the position at which the contact element 43 of the sensor part 20 is positioned. This makes it possible for the contact element 23 to engage in the through-opening 46. The contact element 23 projects in the direction of the tubing 18.

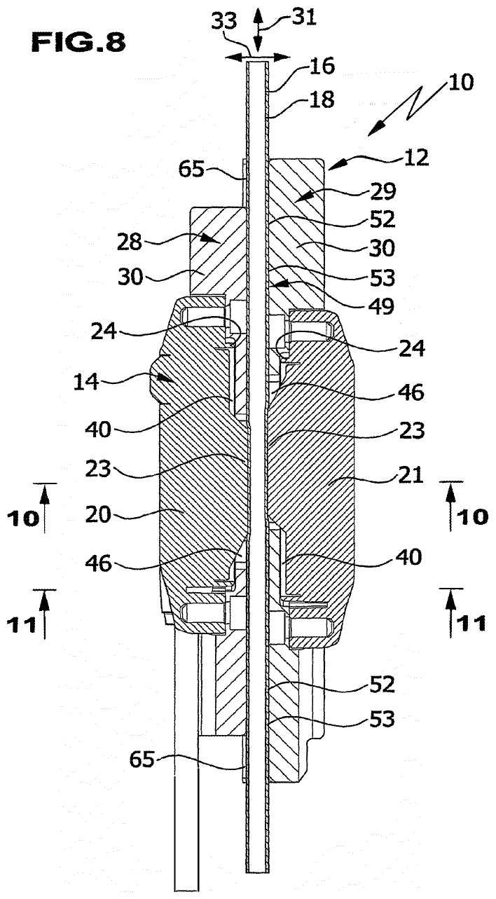

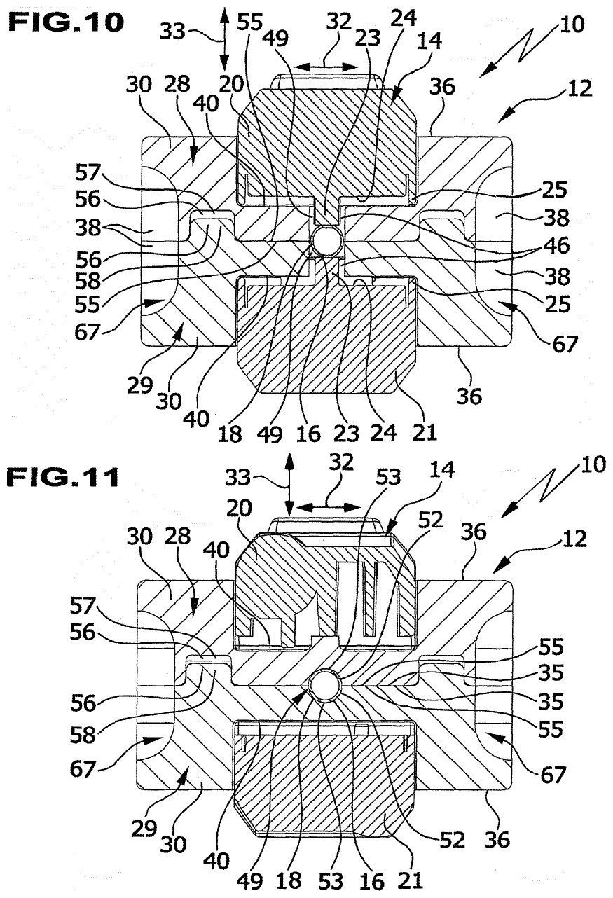

In the present example, the adapter part 28 is dimensioned such that the contact element 23 engages approximately halfway in the through-opening 46 (FIGS. 8 and 10).

In a different preferred embodiment of the disclosure, which is discussed below, the contact element 23 can extend through the through-opening 46.

The sensor part 20 can be rigidly connected to the adapter part 28 and is connected in the state of use. In the present case, connecting elements are used for this purpose. The connecting elements are screw elements 47, which are screwed from the first side 35 through the adapter part 28 to the sensor part 20.

The adapter device 12 comprises a fluid conduit receptacle 49 for receiving the tubing 18. In the present case, the fluid conduit receptacle 49 is formed by the adapter parts 28, 29. The fluid conduit receptacle 49 extends in a straight line and in the direction of extension 31 from a first end face 50 to a second end face 51 of the adapter device 12.

The fluid conduit receptacle 49 comprises grooves 52 on the first side 35 of the respective adapter part 28, 29. The grooves 52 are arranged opposite one another on both sides of the through-opening 46 and directly adjoin the through-opening 46 (FIGS. 5 and 7).

In other words, the design of the fluid conduit receptacle 49 could be described as a continuous groove 52 being provided, also in the region of the through-opening 46, a base of the groove 52 being perforated in the region of the through-opening 46. Such a groove 52 then has two portions adjacent to the through-opening 46, each having a groove base.

Through-openings for the screw elements 47 can be guided through the grooves 52 (FIGS. 5 and 7).

The fluid conduit receptacle 49 and, in particular, the grooves 52 are adapted to the size, in particular the diameter, and to the contour of the tubing 18.

The grooves 52 of the two adapter parts 28, 29 are complementary and are opposite one another in the connection direction 33. The tubing 18 can engage in the grooves 52 on both adapter parts 28, 29, preferably in each case substantially halfway (FIG. 11).

The tubing 18 is held in a defined position between the adapter parts 28, 29 by means of the grooves 52. For this purpose, the grooves 52 each form an abutment region 53 for the tubing 18.

In a plane transverse and, in particular, perpendicular to the direction of extension 31, the grooves 52 have an arc shape and, in particular, the shape of a circular arc, adapted to the size and shape of the tubing 18. An elliptical shape can be provided, as explained below based on the example of FIG. 24.

In a connected state, the adapter parts 28, 29 abut against one another via abutment elements 55 formed by the respective first side 35. In the present case, the abutment elements 55 are planar in order to allow for planar abutment.

Alignment elements 56 on the adapter parts 28, 29 make it possible to clearly define the relative position of the adapter parts 28, 29 in the connected state.

In the present case, two alignment elements 56 are provided on each adapter part 28, 29. In the present example, the adapter part 28 comprises alignment elements 56 in the form of receptacles and the adapter part 29 comprises them in the form of projections. The receptacles are configured as grooves 57, and the projections are configured as strips 58.

An orientation of the adapter parts 28, 29 in the direction of extension 31 and in the transverse direction 32 can take place by means of the alignment elements 56.

The adapter parts 28, 29 can be interconnected and, in particular, can be detachably interconnected. Both the connection and the detachment can conveniently take place manually and, in particular, without tools.

It is advantageous that the adapter device 12 is free of separate connecting elements which engage, in particular, on the outside of the adapter parts 28, 29.

In the present case, the adapter parts 28, 29 are connected by means of magnetic force. The magnetic force is provided by respective magnetic elements 60, which are preferably permanent magnets.

The arrangement of a plurality of magnetic elements 60 on each adapter part 28, 29 is advantageous in order to allow for a reliable connection. In particular, four magnetic elements 60 are provided here in each case.

The adapter part 28 (the same applies to the adapter part 29) comprises receptacles 61 assigned to the magnetic elements 60. The receptacles 61 are configured as blind holes which are formed starting from the second side 36. The magnetic elements 60 are positioned in a positive-locking manner in the receptacles 61. Cover elements 62 close the receptacles 61 and align on the upper side with the second side 36 so that the receptacles 61 are closed substantially without a joining gap.

In the direction of the first side 35, the receptacles 61 are provided with a base in order to prevent dirt from entering the receptacles 61.

In the present case, the magnetic elements 60 are positioned around the sensor receptacle 40 along an imaginary peripheral line. The magnetic elements 60 are arranged substantially in corner regions of the peripheral line, laterally next to corner regions of the sensor receptacles 40. This statement relates to a viewing direction onto the second side 36 of the adapter part 28 or 29.

When interconnecting the adapter parts 28, 29, a clearly defined reproducible relative positioning of said adapter parts can take place by means of the interacting magnetic elements 60 and alignment elements 56.

In the present case, projections 64 are further used for the relative positioning. The projections 64 are arranged on the end faces 50, 51 and protrude from the adapter part 29 over the adapter part 28 in the direction of extension 31.

A groove 65 for receiving the tubing 18 is arranged on each projection 64. The groove 65 is so deep that the tubing 18 can engage completely in the groove 65. In a manner of speaking, an extension of the fluid conduit receptacle 49 beyond the adapter part 28 is thus made possible by means of the groove 65.

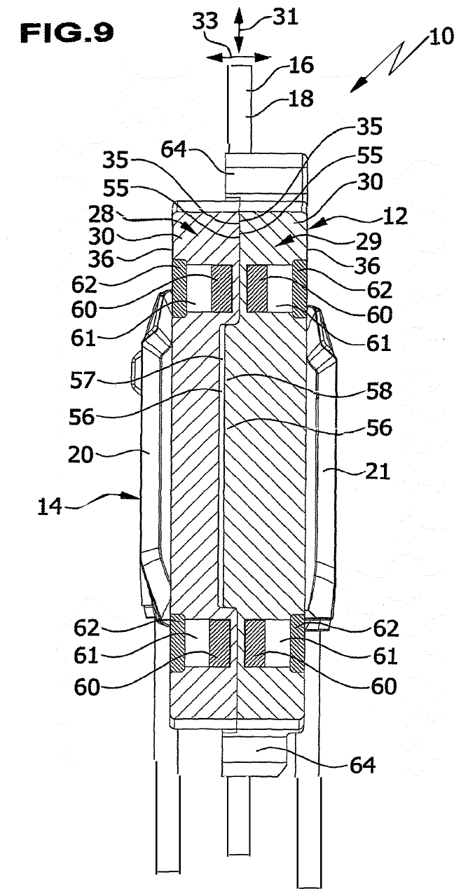

The adapter part 28 engages in the direction of extension 31 in a positive-locking manner between the projections 64 (FIGS. 8 and 9).

In the intended use, the tubing 18 is received in a positive-locking manner in the fluid conduit receptacle 49 and is positioned between the adapter parts 28, 29. Said adapter parts are opposite one another with respect to the tubing 18. Likewise, the sensor parts 20, 21 are opposite one another with respect to the tubing 18. The contact elements 23 are opposite one another with respect to the tubing 18 (FIGS. 8 and 10).

The adapter parts 28, 29 are held against one another by means of the magnetic elements 60.

The contact elements 23 engage in the through-openings 46 (FIGS. 8 and 10), which transition into the fluid conduit receptacle 49. In this way, the contact elements 23 can engage in the fluid conduit receptacle 49. An edge of the through-opening 46 simultaneously delimits the fluid conduit receptacle 49.

The adapter device 12 is dimensioned such that it is adapted to the diameter of the tubing 18. As a result, it can be ensured that the contact elements 23 abut against the tubing 18 in a planar and direct manner, without intermediate elements, preferably without gaps. In particular, a defined pressing force of the contact elements 23 on the tubing 18 can be ensured. This is advantageous for the planar abutment against the tubing 18 (FIGS. 8 and 10).

Due to the positive-locking reception of the tubing 18 not only in the grooves 52 but preferably also in the region of the through-opening 46 between the contact elements 23, there is the possibility of avoiding deformation of the tubing 18 over the course of the service life due to the defined pressing force. The sensor signal provided by the sensor device 14 thus has a high degree of accuracy, even with a longer service life of the measuring device 10.

The sensor receptacles 40 have an offset from one another in the direction of extension 31. As a result, the sensor parts 20, 21 are arranged at different positions in the direction of extension 31 (FIG. 8).

This proves advantageous since the sensor parts 20, 21 radiate the measurement signal (i.e., the emitted ultrasound) at an angle of substantially approximately 450 in the present case. As a result of the offset, as demonstrated in practice, a more accurate measurement can be ensured because the measurement signal is ideally oriented in the direction of the respective other sensor part 20, 21.

In the preferred exemplary embodiment of the method in accordance with the disclosure, it is possible to proceed as follows:

The sensor parts 20, 21 are connected to the adapter parts 28 and 29, respectively. The tubing 18 is arranged in the fluid conduit receptacle 49, for example initially in the grooves 52 of one of the adapter parts 28, 29. The adapter parts 28, 29 are interconnected so that the tubing 18 is arranged between the adapter parts 28, 29. The contact elements 23 directly abut against the tubing 18 without intermediate elements. In particular, the contact elements 18 abut against the tubing 18 without gaps.

The recesses 38 of the adapter parts 28, 29 form engagement regions 67. The engagement regions 67 are, as it were, recessed grips into which the user can conveniently reach in order to detach the adapter parts 28, 29 from one another counter to the magnetic force.

The adapter device 12 is configured to be suitable for pharmaceutical use. Apart from the magnetic elements 60, the components of the adapter parts 28, 29, for example, are manufactured from a material that is suitable for pharmaceutical use, for example stainless steel or POM.

It is advantageous to manufacture the adapter device 12 in such a way that the adapter parts 28, 29 are joined to one another substantially without a joining gap. The outer surface 37, like the sides 35, 36, can be manufactured largely without harsh, abrupt transitions or undercuts. In this way, the adhesion of dirt particles is prevented. The adapter device 12 can be cleaned in a simple manner, in particular also due to the small number of components.

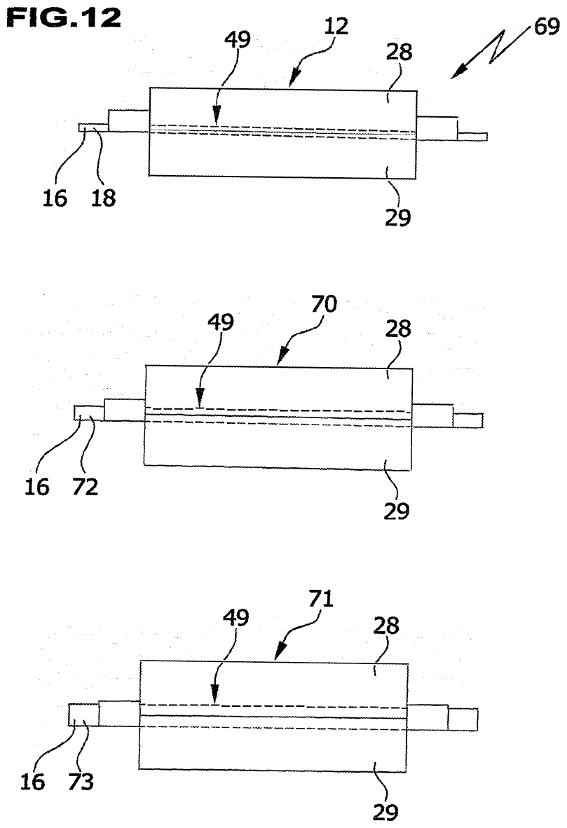

FIG. 12 shows an advantageous embodiment of an adapter arrangement in accordance with the disclosure denoted as a whole by reference numeral 69. In the present case, the adapter arrangement 69 has a first adapter device in accordance with the disclosure, for example the adapter device 12. In addition, the adapter arrangement 69 comprises at least one further adapter device in accordance with the disclosure. In the present case, two further adapter devices 70 and 71 are shown by way of example. They can be functionally identical and structurally identical to the adapter device 12.

In addition, FIG. 12 shows the tubing 18 and further tubing 72 and 73, in each case fluid conduits 16.

The tubing 18, 72 and 73 differ from one another in that they have different sizes. In particular, they have a different diameter. In this case, the tubing 18 is thinner than the tubing 72, which is thinner than the tubing 73.

In the adapter arrangement 69 in accordance with the disclosure, the adapter devices 12, 70 and 71 are each adapted to the size and, preferably, the shape of the tubing 18, 72 and 73. This takes place in that the respective fluid conduit receptacle 49 in the adapter devices 12, 70 and 71 is of a different size and is in each case adapted to the tubing 18, 72 and 73, respectively, in terms of size and, preferably, shape.

Depending on which tubing 18, 72 or 73 is used, the user can select the adapter device 12, 70 or 71 that is best suited for this purpose. The sensor parts 20, 21 (not shown in FIG. 12) can be connected to the adapter parts 28, 29 of the respective adapter device 12, 70 or 71 and used.

The adapter arrangement 69 may also comprise a larger number of adapter devices. Preferably, a separate adapter device 12 is provided for tubing 18 of any diameter. In this case, however, only one sensor device 14 is required, the sensor parts 20, 21 of which can be used with the respective adapter device.

A preferred embodiment of the measuring device in accordance with the disclosure denoted as a whole by reference numeral 75 is discussed below with reference to FIGS. 10 to 17. The measuring device 75 comprises a preferred embodiment of the adapter device in accordance with the disclosure denoted by reference numeral 76. In addition, the measuring device 75 comprises the sensor device 14, the sensor parts 20, 21 of which can be used as explained in connection with the measuring device 10. Reference is made to the above statements, also with regard to the configuration of the adapter parts 28, 29, the relative orientation thereof and the fastening thereof.

A fluid conduit receptacle 49 is also provided in the adapter device 76. In deviation from the adapter device 12, however, the fluid conduit receptacle 49 is not formed by the adapter parts 28, 29 themselves, but rather by an intermediate part 78.

In the present case, the intermediate part 78 has a substantially cuboid basic shape and, in the direction of extension 31, preferably has an extension which coincides with that of the adapter parts 28, 29 from the first end face 50 to the second end face 51.

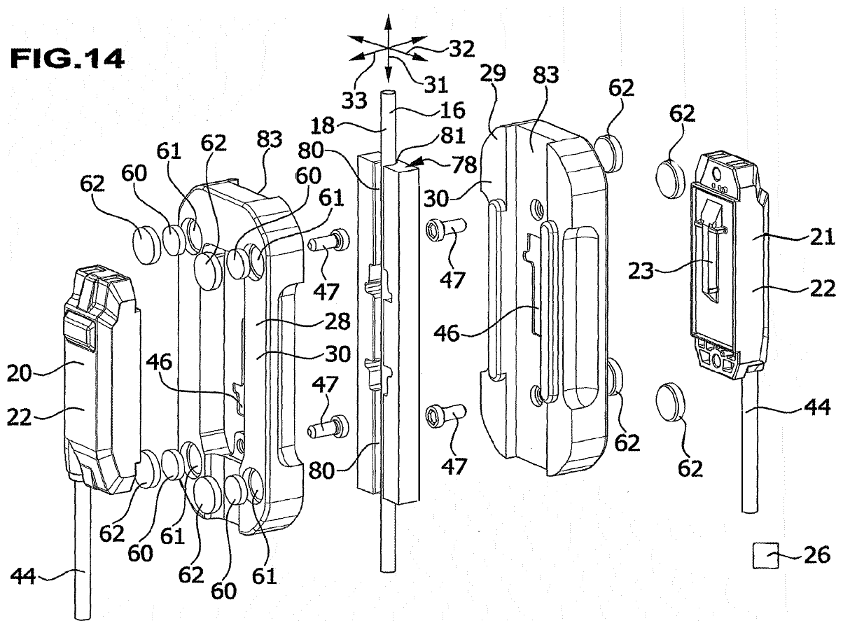

A through-opening 79 is formed in the intermediate part 78. The through-opening 79 is aligned with the through-openings 46 on the adapter parts 28, 29. The shape of the through-opening 79 is obtained by superimposing the shapes of the through-openings 46 of the two adapter parts 28, 29 (FIG. 14).

In the intermediate part 78, a groove 80 is formed in each case directly adjacent to the through-opening 79 in order to receive the tubing 18. In other words, the groove 80 extends over the entire length of the intermediate part 78 and a groove base is perforated in the region of the through-opening 79, as explained by way of example above.

The groove 80 forms abutment regions 81 for the tubing 18, the abutment regions 81 being adapted to the tubing 18 in terms of size and shape. The groove 80 is dimensioned such that the tubing 18 can engage completely therein (FIGS. 14, 15 and 17).

A receptacle 83 oriented in the direction of extension 31 is arranged on the respective adapter part 28, 29 in order to receive the intermediate part 78. In the present case, the intermediate part 78 can engage in a positive-locking manner in the receptacle 83. The intermediate part 78 in each case engages, for example, halfway in the receptacle 83 on the adapter part 28 and on the adapter part 29.

In the transverse direction 32, the receptacle 83 extends between the strips 58.

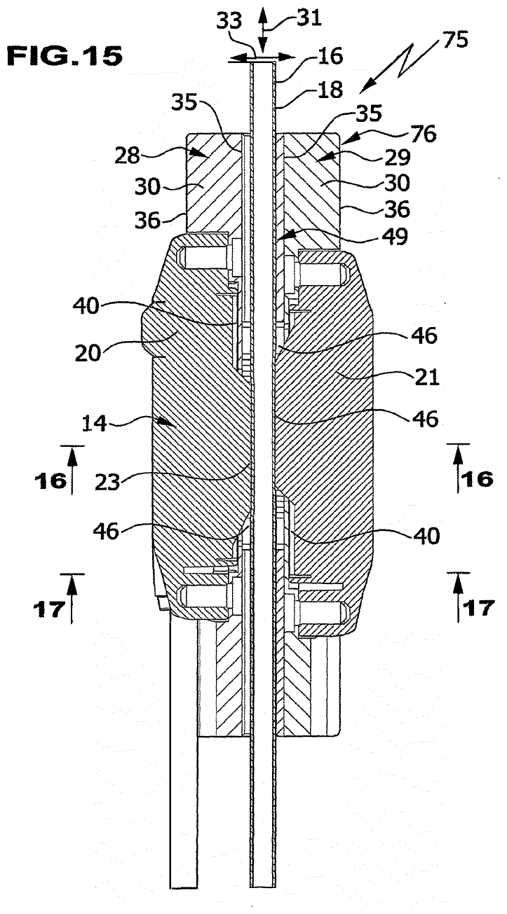

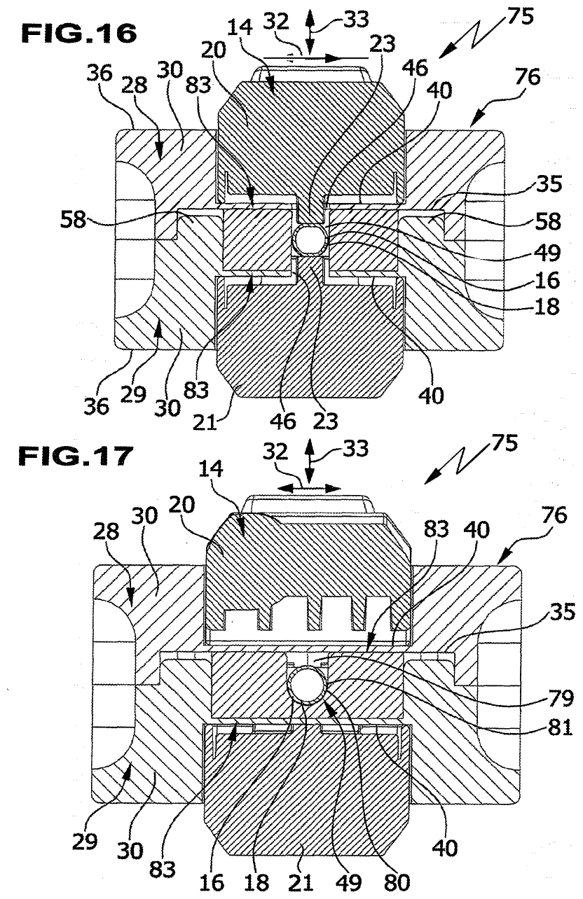

When the measuring device 75 is used as intended, the contact elements 23 extend through the through-openings 46 and engage in the fluid conduit receptacle 49 (FIGS. 15 and 16). The adapter device 76 is dimensioned such that the contact elements 23 can abut against the tubing 18 in a planar manner, directly and without intermediate elements, preferably without gaps and/or with a defined pressing force.

As in the case of the measuring device 10, a pressing force promotes the planar abutment of the contact elements on the tubing 18. The pressing force can be maintained over the course of the service life in order to avoid deformation of the tubing 18 over the course of the service life. As a result, a temporally stable sensor signal is thus possible for a precise measurement of the volumetric flow rate in the tubing 18.

The adapter device 76 has the advantage that the adapter parts 28, 29 can be format-independent, i.e., the identical adapter parts 28, 29 can be used for tubing 18 of different diameters.

In contrast, the intermediate part 78 may be a replacement part (“format part”) adapted to the size and, preferably, the shape of the tubing 18. Depending on which tubing 18 is used, the intermediate part 78 can be exchanged and arranged between the adapter parts 28, 29.

It can be advantageous in the case of the adapter device 76 if the adapter parts 28, 29 can be displaced relative to one another in the direction of extension 31 by an axial offset of the sensor parts 20, 21 with a view to the best possible orientation of the measurement signal toward the respective other sensor part 20, 21. For this purpose, it may also be provided that the adapter parts 28, 29 assume different relative positions in the direction of extension 31.

The adapter device 76 also makes it possible to carry out a preferred exemplary embodiment of the method in accordance with the disclosure:

First, the sensor parts 20, 21 are connected to the adapter parts 28, 29. The tubing 18 is arranged in the fluid conduit receptacle 49 on the intermediate part 78. Subsequently, the intermediate part 78 is arranged between the adapter parts 28, 29 and is secured via the interconnection of the adapter parts 28, 29.

It may be provided in a preferred embodiment that the intermediate part 78 itself comprises magnetic elements and/or magnetizable material which allow/allows for a connection to the adapter part 28 and/or the adapter part 29.

In a different preferred embodiment of the disclosure, it may be provided that the intermediate part 78 is arranged, so to speak, in an overall sandwich-like manner between the adapter parts 28, 29. In this case, the receptacles 83 are omitted, for example, and the intermediate part 78 may extend, for example, in the transverse direction 32 and in the direction of extension 31 over the extension of the adapter parts 28, 29.

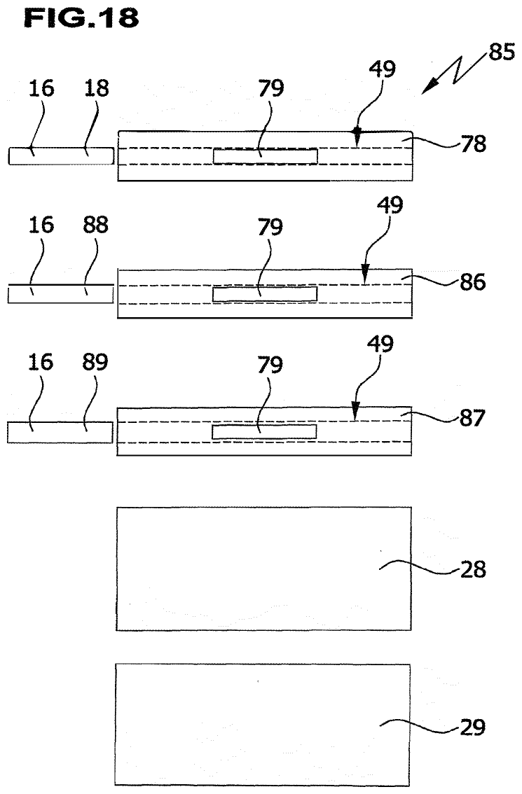

FIG. 18 shows a preferred embodiment of an adapter arrangement in accordance with the disclosure, which is designated by reference numeral 85. The adapter arrangement 85 comprises a preferred embodiment of the adapter device in accordance with the disclosure, in the present case the adapter device 76 having the intermediate part 78.

In addition, the adapter arrangement 85 comprises further intermediate parts, denoted by reference numerals 86 and 87.

In addition to the tubing 18, FIG. 18 shows further tubing 88 and 89, in each case fluid conduits 16. The tubing 18, 88 and 89 differ in terms of their size, in particular their diameter.

The difference between the intermediate parts 78, 86 and 87 is that the respective fluid conduit receptacle 49, in particular the groove 80, is adapted in terms of size and, preferably, in terms of shape to the tubing 18, 88 and 89, respectively. The respective thickness in the connection direction 33 may also depend on the size of the tubing, for example.

Depending on which tubing 18, 88 or 89 is used, the user can select the suitable intermediate part 78, 86 or 87 and insert same between the adapter parts 28, 29. In each case, there is the possibility of placing the contact elements 23 directly with a defined abutment force against the tubing 18, 88 or 89.

The adapter parts 28, 29 are format-independent in the case of the adapter arrangement 85. The intermediate parts 78, 86 and 87 are format parts.

In the present case, the adapter arrangement 85 has three intermediate parts. A different number, in particular at least two intermediate parts, is conceivable here. More than three intermediate parts may be provided.

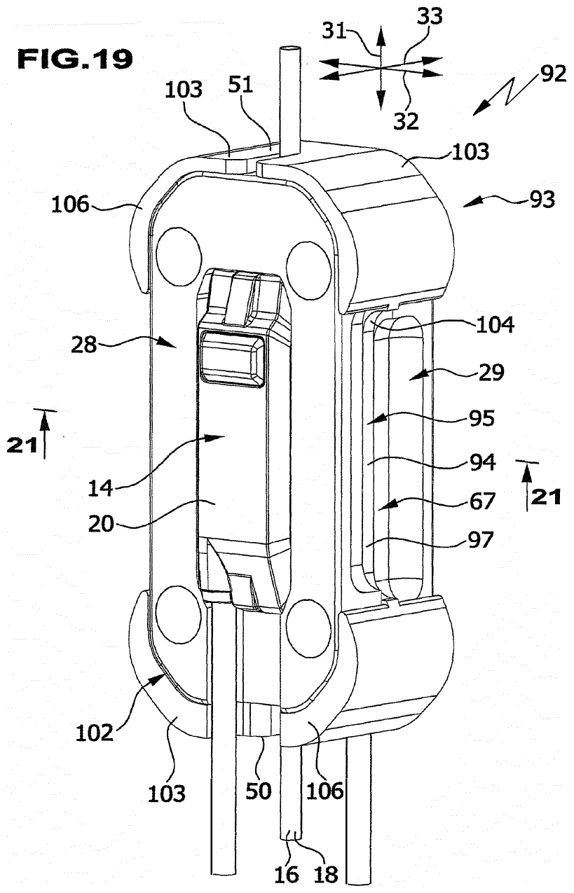

In the following, with reference to FIGS. 19 to 22, a preferred embodiment of the measuring device in accordance with the disclosure denoted as a whole by reference numeral 92 is discussed. The measuring device 92 comprises a preferred embodiment of the adapter device in accordance with the disclosure denoted by reference numeral 93. In addition, the measuring device 92 comprises the sensor device 14, the sensor parts 20, 21 of which can be used as explained in connection with the measuring device 10. Reference is made to the above statements, also with regard to the configuration of the adapter parts 28, 29.

In the case of the adapter device 93, an intermediate part is also provided, which is denoted by reference numeral 94 and which forms the fluid conduit receptacle 49. In addition, the intermediate part 94 in the adapter device 93 is a receiving part 95 for the two adapter parts 28, 29.

The intermediate part 94 has a substantially plate-shaped portion 97. In the connected state, the adapter parts 28, 29 receive the plate-shaped portion 97 between them and preferably abut against it in a planar manner. The alignment elements 56 are omitted. The adapter parts 28, 29 are held against one another by means of the magnetic elements 60. In addition, it can be provided, for example, that the receiving part 95 comprises magnetic elements and/or a magnetizable material, for example on the portion 97, in order to ensure a reliable connection to the adapter parts 28, 29 and/or the adapter parts 28, 29.

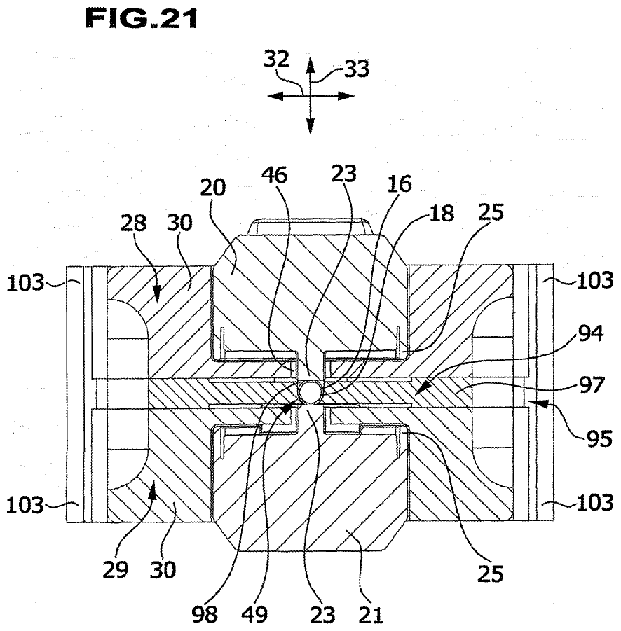

A through-opening 98 is formed on the portion 97 and is aligned at least in portions with the through-openings 46 (FIG. 21). The fluid conduit receptacle 49 has grooves 99 for the tubing 18 that are opposite one another with respect to the through-opening 98 and that are directly adjacent thereto. The groove 99 forms a respective abutment region 100 for the tubing 18.

As explained in the preceding exemplary embodiments, this design can be described as a continuous groove 99 having an abutment region 100 for the tubing 18 being provided, a base of the groove 99 being perforated in the region of the through-opening 98.

The abutment regions 100 are adapted to the tubing 18 in terms of size and shape. The groove 99 is dimensioned such that the tubing 18 can engage completely therein, as has been described above based on the example of the intermediate part 78.

As explained in the example of the adapter device 76, the adapter device 93 also makes it possible for the contact elements 23 to extend through the through-openings 46. The contact elements 23 can be placed directly against the tubing 18, preferably in a planar manner and without gaps. A defined pressing force is advantageously provided (FIG. 21).

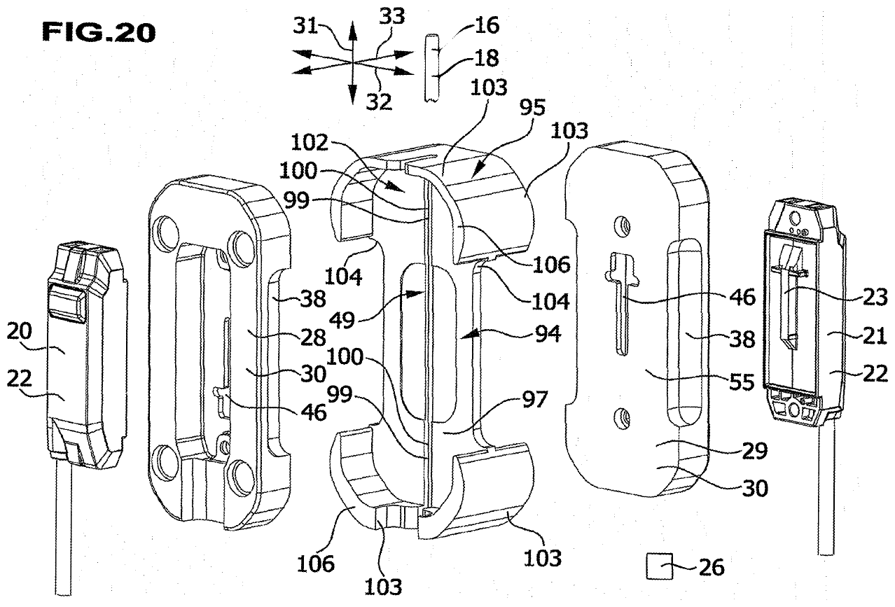

As can be seen, in particular, from FIGS. 19 and 20, the receiving part 95 comprises a respective adapter part receptacle 102 for the first adapter part 28 and for the second adapter part 29. The adapter part receptacles 102 are each dimensioned such that the adapter parts 28, 29 can be arranged in a positive-locking manner therein.

Wall arrangements 103 facing the adapter part 28 protrude from the portion 97. The wall arrangements 103 are spaced apart from one another in the direction of extension 31 so that the adapter part 28 is positioned between the wall arrangements 103 (FIG. 19).

Accordingly, the portion 97 projects beyond the adapter parts 28, 29 in the direction of extension 31 and, in the present case, also in the transverse direction 32 on both sides of the adapter device 93, the wall arrangements 103 protruding from the portion 97.

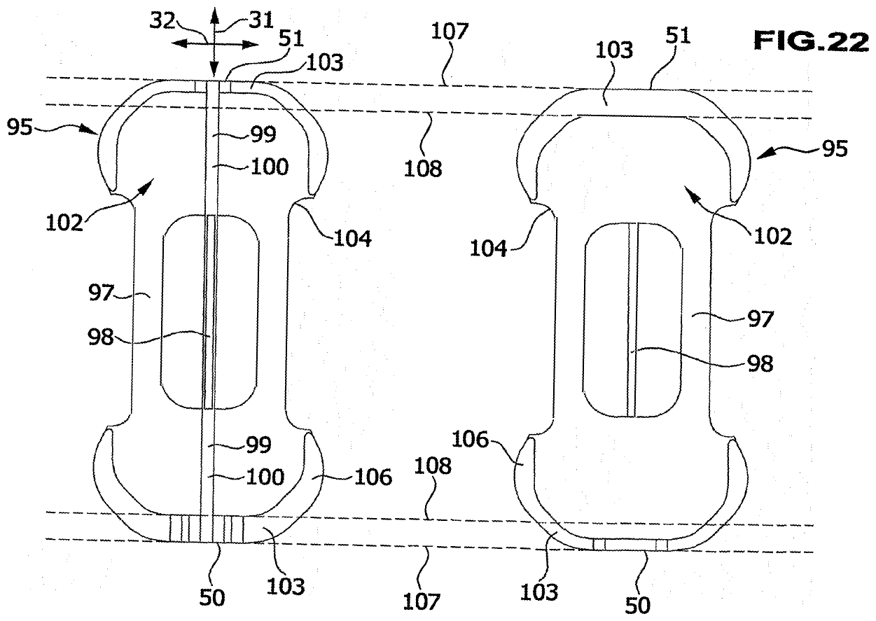

The respective wall arrangement 103 is arranged in the region of the end faces 50, 51. Proceeding from the end faces 50, 51, the wall arrangement 103 extends in the direction of the outer sides of the adapter device 93 that are spaced apart from one another in the transverse direction 32. A respective wall arrangement 103 is substantially C-shaped in plan view (FIG. 22).

Recesses 104 that are spaced apart from one another in the transverse direction 32 are also formed in the receiving part 95. In the connected state, the recesses 104 are part of the engagement regions 67.

In a plan view in the connection direction 33, the receiving part 95 is substantially bone-shaped (FIG. 22).

The wall arrangements 103 are rounded along the outer periphery of the adapter device 93. This proves advantageous for cleaning.

In the connection direction 33, the height of the adapter part receptacle 102 is dimensioned such that the second side 36 is aligned with an edge 106 of the wall arrangement 103 facing away from the portion 97.

The same applies to the adapter part receptacle 102 for the adapter part 29. This adapter part receptacle 102 also comprises the wall arrangements 103 as described above. The respective wall arrangements 103 of the two adapter part receptacles 102 are aligned with one another in the connection direction 33 (FIGS. 19 and 20).

Recesses in the wall arrangements 103 on the end faces 50 and 51 make it possible for the tubing 18 to be guided through.

FIG. 22 shows the receiving part 95 in two different plan views, the plan view on the left showing the adapter part receptacle 102 for the adapter part 28 and the plan view on the right showing the adapter part receptacle 102 for the adapter part 29.

It is evident from FIG. 22 that the adapter part receptacles 102 have an offset relative to one another in the direction of extension 31. Dashed lines 107 indicate the outer boundaries of the receiving part 95 on the end faces 50 and 51. Dashed lines 108 indicate the thickness of the respective thicker wall arrangement 103 in the region of the end face 50 and the end face 51. It is evident that the thicker wall arrangement 103 is provided in the region of the end face 50 in the case of the adapter part receptacle 102 for the adapter part 28 and in the region of the end face 51 in the case of the adapter part receptacle 102 for the adapter part 29. In the case of the respective thinner wall arrangements 103, this is reversed, as can be seen from FIG. 22.

The offset of the adapter part receptacles 102 can ensure the already explained offset of the adapter parts 28, 29 and thus of the sensor parts 20, 21. This offers the advantage of a better signal with a view to a more precise measurement of the volumetric flow rate through the tubing 18.

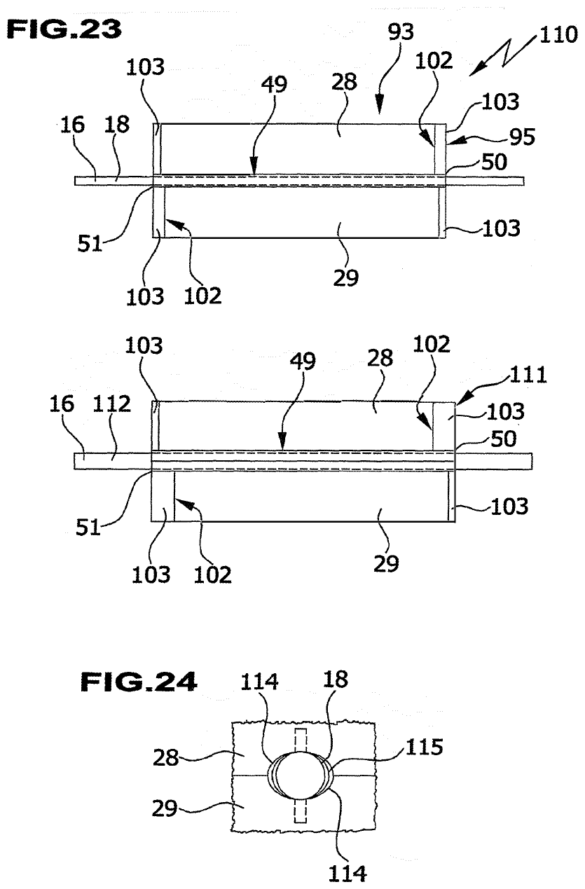

FIG. 23 shows an embodiment of an adapter arrangement in accordance with the disclosure, which is designated as a whole by reference numeral 110. The adapter arrangement 110 comprises a preferred embodiment of the adapter device in accordance with the disclosure, preferably the adapter device 93 having the receiving part 95.

In addition, the adapter arrangement 110 comprises at least one further receiving part, denoted by reference numeral 111. It is understood that more than one further receiving part 111 may be provided and the disclosure is not limited to this number.

In addition to the tubing 18, FIG. 23 shows further tubing 112, in turn a fluid conduit 16. The tubing 18 and 112 differ in terms of their size, in particular their diameter.

The difference between the receiving parts 95 and 111 is that the respective fluid conduit receptacle 49, in particular the groove 99, is adapted in terms of size and, preferably, in terms of shape to the tubing 18 or 112. In this case, the larger the diameter, the thicker the respective portion 97 (in the connection direction 33).

This ensures that the contact elements 23 can each be placed directly against the inserted tubing 18 by means of a suitable pressing force.

The receiving parts 95 and 111 further differ in that the offset of the adapter part receptacles 102 is different. In particular, the larger the diameter of the tubing 18, the greater the offset of the adapter parts 28, 29 and thus of the sensor parts 20, 21 in the direction of extension 31. This offers the advantage already mentioned that the measurement signal is ideally oriented toward the respective other sensor part 20, 21 with a view to a measurement of the volumetric flow rate that is as precise as possible when the diameter of the tubing 18 is different.

In FIG. 23, the offset can be discerned from the thickness of the wall arrangements 103, the wall thickness in FIG. 23 at the top being shown based on the example of the receiving part 95 as shown in FIG. 22. The situation at the bottom of FIG. 23 shows a thicker wall arrangement 103 on each adapter part receptacle 102.

As already mentioned above, the respective receiving part 95, 111 is an intermediate part of the adapter device.

The receiving parts 95 and 111 are so-called “format parts.” The adapter parts 28, 29 are format-independent. In particular, the sensor parts 20, 21 can remain mounted on the adapter parts 28, 29 when the receiving part 95, 111 is changed.

It has already been mentioned above that tubing, in particular, can have dimensional tolerances.

In order to counteract dimensional tolerances, it may be provided that a cross section of the fluid conduit receptacle 49, for example the groove 52, 80 and/or 99, deviates from a circular shape. The cross section may, for example, be elliptical. It can be provided here that a clear width of the fluid conduit receptacle 49 in the transverse direction 32 is greater than a clear width in the connection direction 33. FIG. 24 schematically represents a corresponding situation.

In FIG. 24, reference numeral 114 denotes a respective groove of the fluid conduit receptacle 49 on the adapter parts 28, 29, which groove could be provided in each of the adapter devices described here. Reference numeral 18 denotes tubing having a circular shape, more or less without a dimensional tolerance.

In contrast, reference numeral 115 denotes tubing that has a non-negligible dimensional tolerance. Due to the elliptical shape of the groove 114, the tubing 115 can be deformed as a result of the pressing force of the contact elements 23. The tubing 115 expands in the width direction (transverse direction 32).

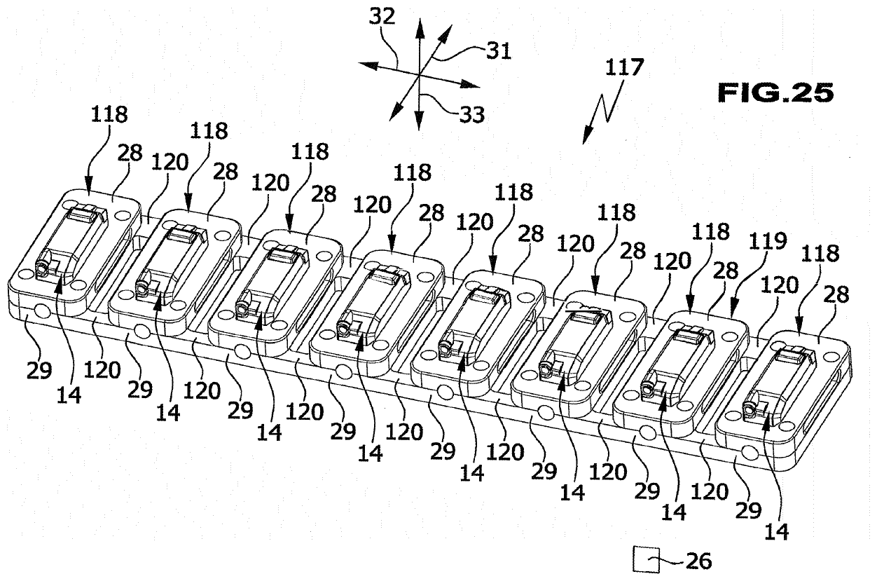

FIG. 25 shows a preferred embodiment of the measuring device in accordance with the disclosure denoted by reference numeral 117 and having a plurality of adapter devices 118 and the respective sensor devices 14 assigned thereto. Fluid conduits, in particular tubing, are not shown in FIG. 25.

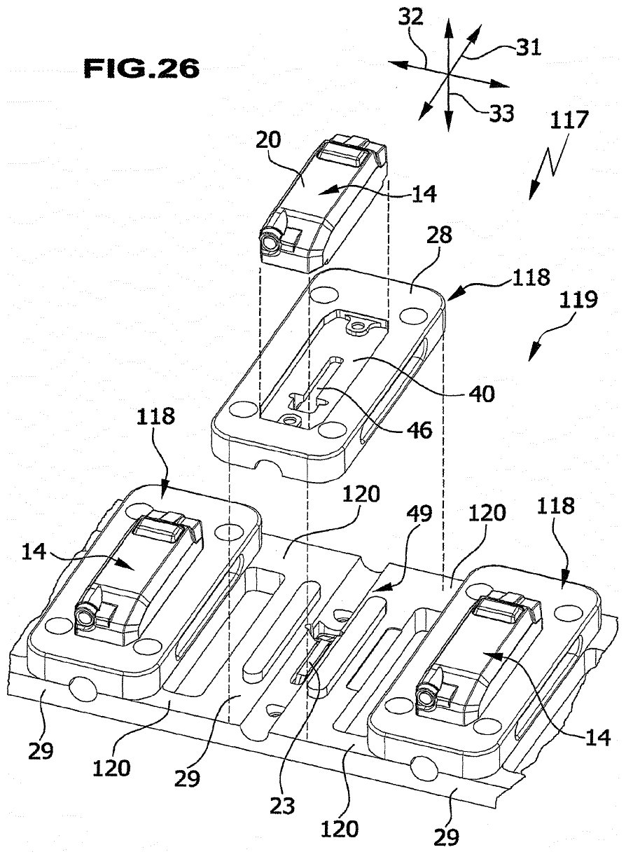

FIGS. 25 and 26 further show a preferred embodiment of the adapter system in accordance with the disclosure which is designated by reference numeral 119 and comprises the adapter devices 118. The adapter system 119 may be part of the measuring device 117.

The adapter device 118 is, for example, largely identical to the adapter device 12. The projections 64 are missing at the end faces 50, 51. In functional terms, there are substantially no differences between the adapter devices 12 and 118, except for fluid conduit receptacles 49 of different sizes.

In the present example, the second adapter parts 29 are interconnected in the adapter system 119. Connecting elements 120 are arranged between adjacent adapter parts 29. In the present case, the connecting elements 120 are web-shaped and arranged in the region of the end portions of the adapter parts 29 arranged in the direction of extension 31. The connection of the adapter parts 29 is preferably in one piece, it being possible for the connecting elements 120 to be integrally formed thereon.

In contrast, the adapter parts 28 are not interconnected in the adapter system 119 but rather are formed separately from one another.

The adapter devices 118 are positioned laterally next to one another, in particular in a straight row next to one another. In the transverse direction 32, the adapter devices 118 are arranged next to one another and at an equal distance from one another. The fluid conduit receptacles 49 are aligned in parallel with one another.

The adapter system 119 can be used, for example, in so-called multistation filling stations, in which a plurality of filling elements are provided, to each of which a fluid conduit is connected. An adapter device 118 having a sensor device 14 is arranged on each fluid conduit in order to measure the flow through the respective fluid conduit. In the present example, the adapter system 119 has eight adapter devices 118, the number of which could also vary.

The respective adapter part 28, as can be seen in particular from FIG. 26, can be connected to and detached from the adapter part 29 assigned thereto separately from the adapter parts 28. In this way, the respective fluid conduit receptacle 49 with the fluid conduit 16 arranged therein can be closed or exposed.

In the adapter system 119, the adapter parts 28 and 29 are adapted to the sizes and, in particular, to the diameter of the fluid conduit and are format-dependent. Different adapter systems 119 are used with different sizes of the fluid conduit.

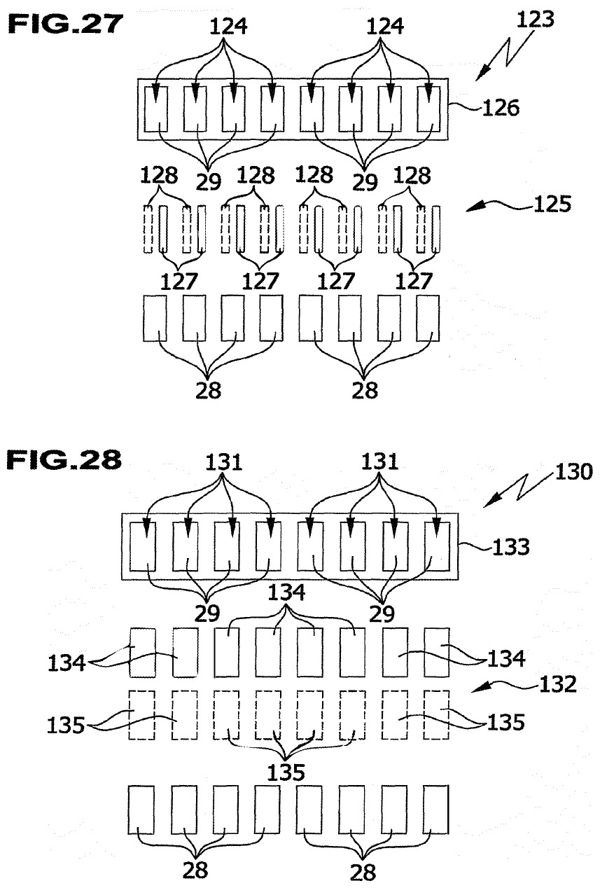

FIG. 27 schematically shows an adapter system in accordance with the disclosure in a preferred embodiment, which comprises a plurality of adapter devices 124 in accordance with the disclosure. In the present case, the adapter system 123 also comprises a plurality of adapter arrangements 125 in accordance with the disclosure, which are designed, for example, to correspond to the adapter arrangement 85 or to be functionally identical thereto, as far as the presence of a plurality of intermediate parts is concerned.

The second adapter parts 29 are interconnected, this being schematically illustrated in FIG. 27 by a solid line 126. The connection can be formed, for example, as in the adapter system 119. The adapter parts 28 are formed separately from one another, as in the case of the adapter system 119.

An intermediate part 127 and at least one further intermediate part 128 are provided in each case in the adapter arrangement 125, said further intermediate part being represented in FIG. 27 by a dashed line.

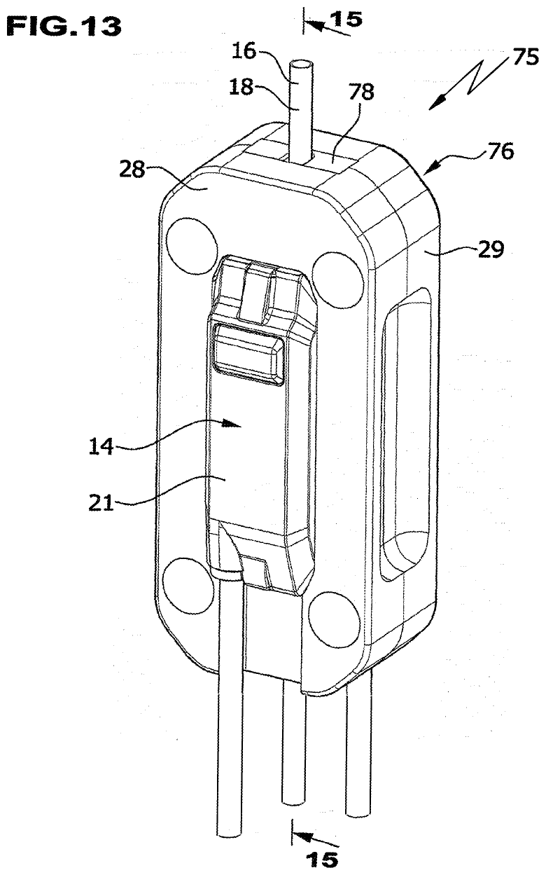

The intermediate parts 127, 128 are adapted to size and/or shape, in particular to the diameter, of the respective fluid conduit 16. The intermediate parts 127, 128 are format parts. In contrast, the adapter parts 28, 29 are format-independent. Reference is made to the above statements regarding FIGS. 13 to 18 and, in particular, with respect to the adapter arrangement 85.

FIG. 28 is a schematic representation of a preferred embodiment of an adapter system in accordance with the disclosure, designated as a whole by reference numeral 130. The adapter system 130 comprises a plurality of adapter devices 131. In the present case, the adapter system 130 also comprises a plurality of adapter arrangements 132 in accordance with the disclosure, which are designed, for example, like the adapter arrangement 110 or so as to be functionally identical thereto, as far as the presence of a plurality of receiving parts is concerned.

The second adapter parts 29 are interconnected and are indicated by a solid line 131 in FIG. 28. The connection is, for example, such as has been described for the adapter system 119. In contrast, the adapter parts 28 are formed separately from one another, as in the case of the adapter system 119.

The adapter system 130 comprises a receiving part 134 having at least one adapter receptacle 102. In the present case, the adapter system 130 comprises at least one further receiving part 135, which is shown as a dashed line in FIG. 28. The receiving parts 134, 135 are adapted to the size and/or shape, in particular to the diameter, of the fluid conduit 16. In the present case, the receiving parts 134, 135 are format parts. The adapter parts 28, 29 are format-independent.

Furthermore, reference is made to the statements regarding FIGS. 19 to 23, in particular with respect to the adapter arrangement 110.

In the adapter system 123, preferably two or more intermediate parts 127, 128 are used, the adapter system 130 preferably comprising two or more receiving parts 134, 135.

It is understood that, in deviation from the above explanations, adapter systems 123, 130 in accordance with the disclosure can be provided with only one intermediate part 127 and only one receiving part 134, respectively.

In the case of the adapter system 123 and the adapter system 130, it could even be provided that different intermediate parts 127, 128 and different receiving parts 134, 135, respectively, can be used in the adapter devices 124 and 131, respectively. This possibility allows fluid conduits 16 of different sizes and, in particular, of different diameters to be attached to the adapter system 123 or 130. Since the adapter parts 28, 29 are in each case format-independent, the adaptation to the fluid conduits 16 preferably takes place solely by means of the format-dependent intermediate parts 127, 128 or receiving parts 134, 135.

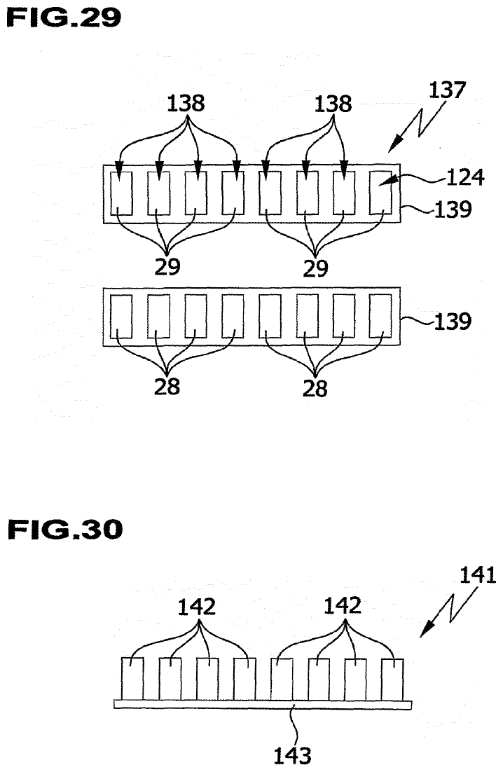

FIG. 29 shows a preferred adapter system 137 in a preferred embodiment having a plurality of adapter devices 138. In the adapter system 137, both the adapter parts 28 and the adapter parts 29 are interconnected, indicated in each case by a solid line 139. The connection takes place, for example, as described with respect to the adapter system 119. The adapter parts 28 and the adapter parts 29 can in each case be connected together.

FIG. 30 shows a preferred embodiment of an adapter system 140 in accordance with the disclosure. The adapter system comprises a plurality of adapter devices 142. The adapter devices 142 are interconnected via a common holding part 143. The holding part 143 is, for example, a strip by means of which the adapter system 141 can be fastened to a supporting part of the pharmaceutical plant.

It is understood that, insofar as this has not been explained in detail, the adapter system in accordance with the disclosure, the adapter arrangements in accordance with the disclosure or the adapter device in accordance with the disclosure can each comprise a holding part for securing to a supporting part of the pharmaceutical plant. A respective holding part may be part of the measuring device in accordance with the disclosure.

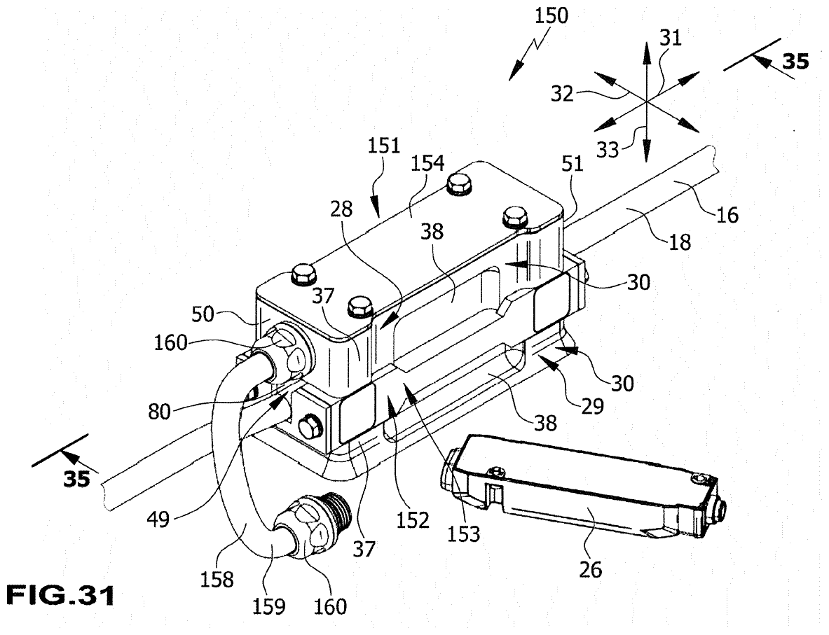

Another preferred embodiment of the measuring device in accordance with the disclosure is shown in FIG. 31 and is denoted by reference numeral 150. The measuring device 150 comprises a preferred embodiment of the adapter device in accordance with the disclosure with reference numeral 151. It is predominantly the main differences with respect to the preceding measuring devices and adapter devices that are discussed. Otherwise, the above statements apply mutatis mutandis.

The adapter device 151 comprises an intermediate part 152 which can be positioned between the adapter parts 28, 29 and which forms the fluid conduit receptacle 49. In the present case, the intermediate part 152 forms a receiving part 153.

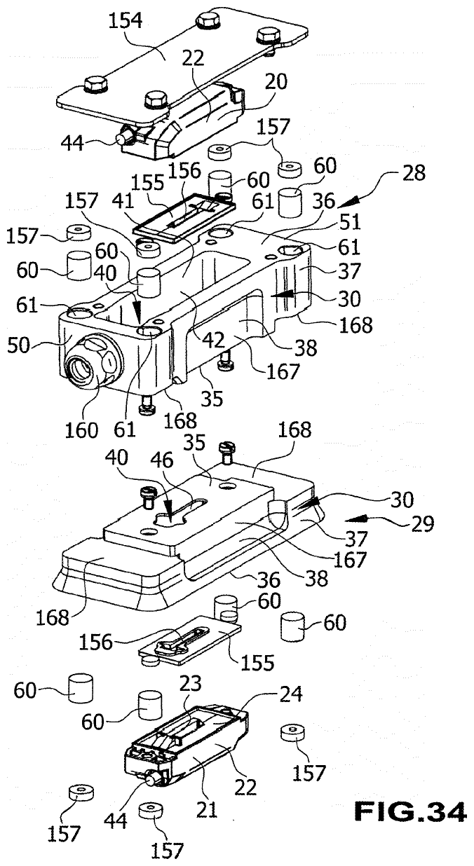

As can be seen, in particular, from FIG. 34, in the first adapter part 28, on the second side 36, a cover element 154 covering the latter is provided. The cover element 154 also covers the opening of the sensor receptacle 40 and protects the sensor part 20 from external influences. The cover element 154 is connected to the base body 30, for example by means of screwing.

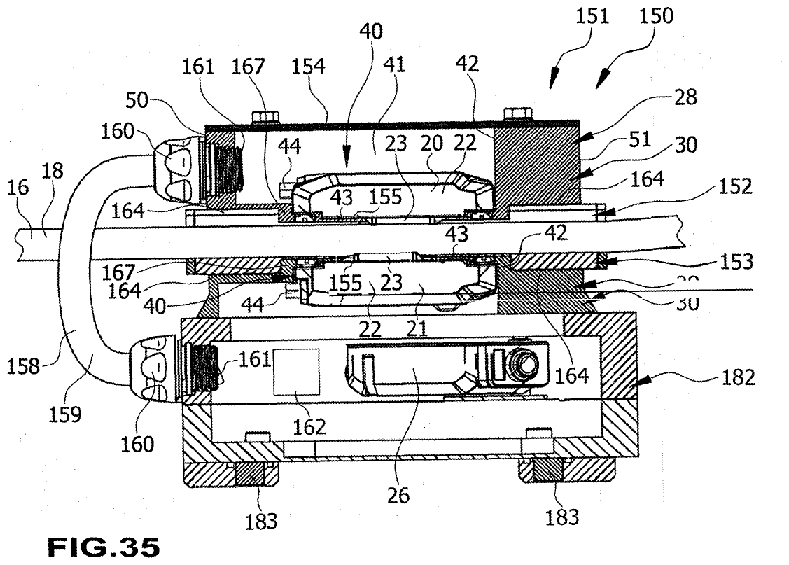

The sensor part 20 abuts against the abutment region 43 of the sensor receptacle 40 via a sealing element 155 (FIG. 35). The sealing element 155 comprises a cutout 156 which is aligned with the through-opening 46. The contact element 23 extends through the cutout 156 and engages in the through-opening 46 or extends therethrough, as already explained.

In a corresponding manner, a sealing element 155 is arranged between the sensor part 21 and the abutment region 43 on the second adapter part 29.

The sealing elements 155 preferably prevent the ingress of liquid into the sensor receptacle 40 in the event of a damaged fluid conduit 16.

In the case of the adapter device 151 as well, the magnetic elements 60 are provided in corresponding receptacles 61 of the base body 30 in order to hold the adapter parts 28, 29 in a connected state in which the intermediate part 152 is arranged in a sandwich-like manner between the adapter parts 28, 29. Buffering pressing elements 157 are positioned above the magnetic elements 60 in the receptacles 61 and, in this way, prevent the magnetic elements 60 from moving. The receptacles 61 are closed by means of the cover element 154.

As can be seen, in particular, from FIGS. 31 and 35, the adapter device 151 comprises a connecting part 158. In the present case, the connecting part 158 comprises a deformable tube element 159. In the present case, the tube element 159 is connected to the adapter parts 28 on the end face 50 thereof. For this purpose, a screw element 160 is provided, which is screwed to a bushing 161 inserted into a through-opening of the base body 30.

The connection line 44 is guided through the tube element 159 (this can also be provided for the connection line 44 of the sensor part 21) and electrically connected to the evaluation device 26 or indirectly to the evaluation device 26 via an electrical distributor element 162.

The connecting part 158 is further connected to a holding part (182), which may be part of the adapter device 151 or, as explained below, part of an adapter system in accordance with the disclosure.

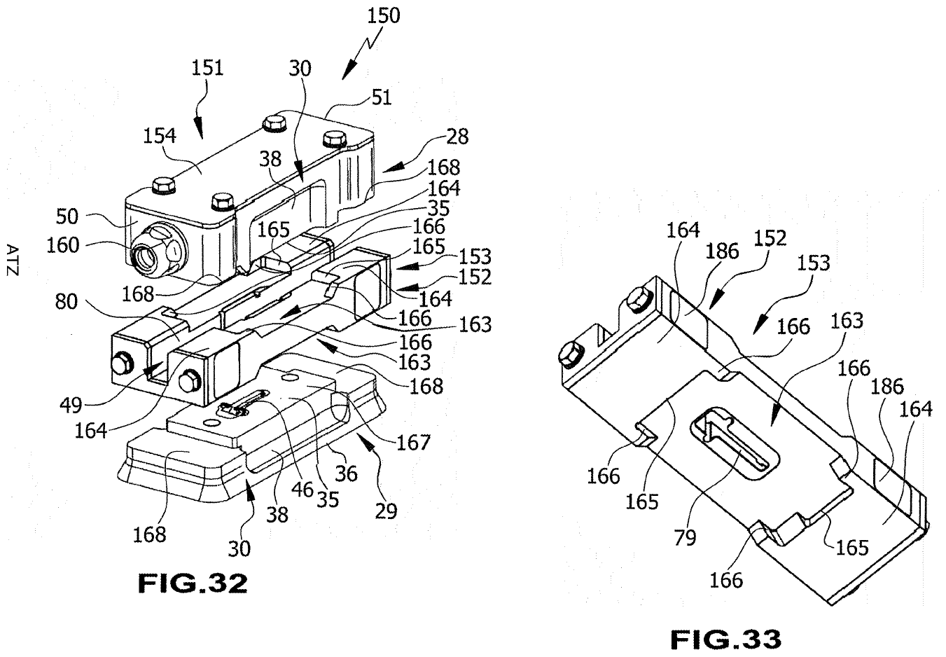

As can be seen, in particular, from FIGS. 31 to 33, in the adapter device 151, the adapter parts 28, 29 and the intermediate part 152 are each provided with a stepped shape, in relation to the extension direction 31. In the present example, the intermediate part 151 is substantially block- or plate-shaped.

A receptacle 163 that is trough-shaped in the present example on account of a depression and that acts as an adapter part receptacle in which the respective adapter part 28, 29 engages in portions is formed in the intermediate part 152 so as to face a respective adapter part 28, 29. A raised portion 164 is arranged laterally (in the direction of the end faces 50 and 51) next to the receptacle 163 in relation to the direction of extension 31.

The portions 164 and the receptacle 163 are interrupted centrally in the transverse direction 32 by the fluid conduit receptacle 49. The fluid conduit receptacle 49 is formed by a groove 80, which, in the present example, has a rectangular cross section.

The edges 165 in the transverse direction 32 and 166 in the direction of extension 31 allow for clear relative positioning of the respective adapter part 28, 29 relative to the intermediate part 152.

In the direction of extension 31, the adapter parts 28, 29 each centrally have a projecting portion 167, laterally next to which recessed portions 168 (which are recessed in the direction of the end faces 50 and 51) are arranged. The through-openings 46 are formed in the projecting portions 167. The projecting portion 167 is, for example, substantially platform-shaped.

The projecting portions 167 engage in a positive-locking manner in the receptacles 163 so that the position of the adapter parts 28, 29 with respect to the intermediate part 152 is clearly defined in the direction of extension 31 and in the transverse direction 32.

The respective raised portions 164 abut against the recessed portions 168.

Advantageously, a planar abutment of the projecting portion 167 against the base of the receptacle 163 and/or a planar abutment of the raised portion 164 against the recessed portion 168 is provided.

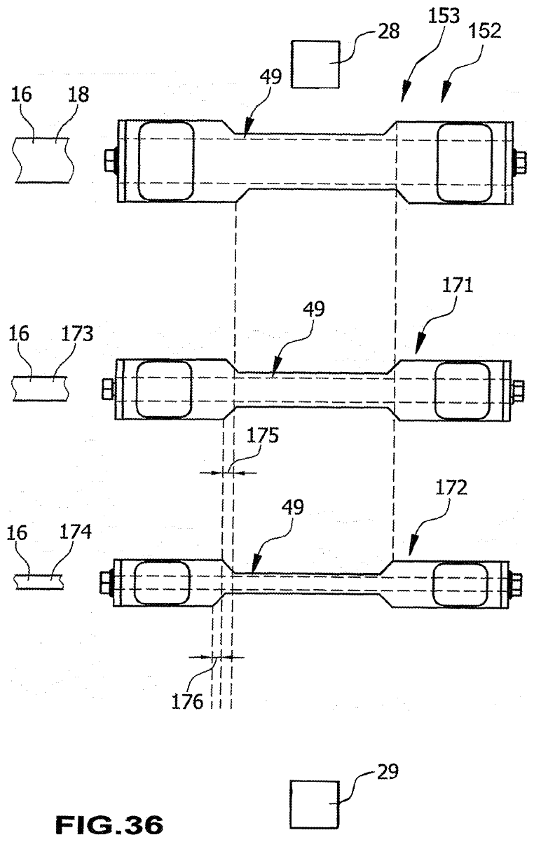

As is clear from FIGS. 35 and 36, for example, the receptacles 163 of the intermediate part 152 have an offset in the direction of extension 31. In this way, the measurement result can be improved for the sensor parts 20, 21 used, as has already been explained.

In the present case, the intermediate part 152 comprises magnetizable elements, for example made of steel, which interact with the magnetic elements 60 for connection to the adapter parts. As a result, a magnetic holding force can act between each adapter part 28, 29 and the intermediate part 152 for the respective connection thereof.

Reference numeral 186 in FIG. 33 denotes, for example, a cover element for closing a receptacle in the intermediate part 152 into which the magnetizable element is inserted. Alternatively, casting or injection of the magnetizable element or a different type of sealing process is conceivable.

FIG. 36 shows schematically, in a manner similar to FIG. 23, an adapter arrangement in accordance with the disclosure with reference numeral 170. The adapter arrangement 170 comprises a preferred embodiment of the adapter device in accordance with the disclosure, for example the adapter device 151 having the intermediate part that forms the receiving part 153.

In addition, the adapter arrangement 170 comprises at least one further receiving part. FIG. 36 shows, by way of example, two further receiving parts 171 and 172, which are in particular intermediate parts.

Further provided in FIG. 36 are the tubing 18 and further fluid conduits in the form of tubing 173, 174, which have a smaller diameter than the tubing 18, the diameter of the tubing 173 being larger than in the case of the tubing 174.

As already explained above based on the example of the adapter arrangement 110, the respective fluid conduit receptacle 49 is adapted in terms of size and/or shape to the tubing 18, 173 or 174.

It can also be seen that the offset of the receptacles of the respective receiving part 153, 171 and 172 depends on the diameter of the fluid conduit 16 to be inserted. Reference numeral 175 indicates an offset between the receptacles 163 of the receiving parts 153, 171 and reference numeral 176 indicates the offset between the receiving parts 171 and 172. The larger the diameter, the greater the offset.

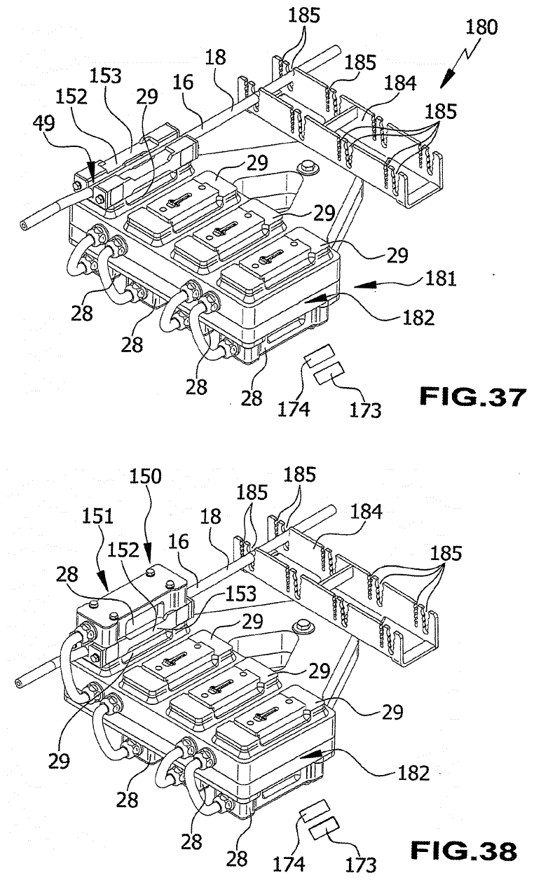

FIGS. 37 and 38 show a preferred embodiment of a measuring device 180 in accordance with the disclosure, which comprises a preferred embodiment of an adapter system 181 in accordance with the disclosure. The adapter system 181 in turn has adapter devices in accordance with the disclosure for multistation operation, in the present case the adapter device 151 in a multiple amount. In addition, the adapter system may comprise an adapter arrangement in accordance with the disclosure having further intermediate parts and/or receiving parts. By way of example, the receiving parts 171, 172 of the adapter arrangement 170 are provided.

The adapter system 181 comprises a holding part 182. In the present example, the two adapter parts 29 are interconnected by means of the holding part 182. The holding part 182 forms, for example, a frame (FIG. 35) on which the adapter parts 29 are secured. The adapter parts 29 are positioned laterally next to one another.

In the present example, the holding part 182 receives the evaluation device 26 and, if present, the electrical distributor element 162.

Only one intermediate part 152 of the intermediate parts 153 is shown in FIGS. 37 and 38. It is understood that the other adapter devices 151 also comprise an intermediate part. The intermediate part 152 is magnetically connected to the adapter part 29.

The adapter system 181 makes it possible to transfer the adapter parts 28 into a maintenance state. In FIG. 37, all adapter parts 28 assume the maintenance state.

In the maintenance state, the adapter parts 28, 29 are detached from one another, and the fluid conduit receptacle 49 is released. This serves, for example, for cleaning purposes or for exchanging the fluid conduits 16 and/or the intermediate parts 152.

The adapter part 28 is detachably secured to the holding part 182 in order to assume the maintenance state. For this purpose, connecting elements are preferably provided on the holding part 182, in the present case in the form of magnetic elements or magnetizable elements (for example made of steel) 183 (FIG. 35). In this way, the adapter part 28 can conveniently be magnetically connected to the holding part 182.

In order to assume the connected state, the adapter part 28 is detached from the holding part 182, positioned on the intermediate part 152 and connected thereto (FIG. 38).

It is advantageous if the connecting part 158 is provided. As a result, the adapter part 28 is held captively on the holding part 182, so to speak. In addition, the guidance for the connection line 44 is ensured.

Advantageously, a guide part 184 can be provided in the adapter system 181. The guide part 184 is used for guiding the fluid conduits 16. Advantageously, for this purpose, recesses 185 of different widths are provided, which are suitable for receiving different fluid conduits 16 and allow for a reliable fit of the fluid conduits 16. The guide part 184 is preferably connected to the holding part 182.

1. An adapter device for attaching a sensor device having a first sensor part and a second sensor part to a pharmaceutical fluid conduit that has a direction of extension, the adapter device comprising a first adapter part and a second adapter part, the first adapter part comprising or forming a first sensor receptacle for the first sensor part and the second adapter part comprising or forming a second sensor receptacle for the second sensor part, the adapter parts being directly or indirectly interconnectable, and also a fluid conduit receptacle which is arranged between the adapter parts or is formed by the adapter parts and which is designed to receive the fluid conduit, the first sensor receptacle being arranged on a side of the first adapter part facing away from the fluid conduit receptacle and the second sensor receptacle being arranged on a side of the second adapter part facing away from the fluid conduit receptacle, a respective through-opening being formed on the first sensor receptacle and on the second sensor receptacle, and a respective contact element of the first sensor part and of the second sensor part engaging in the respective through-opening or extending therethrough for direct abutment against the fluid conduit in a planar manner.

2. The adapter device in accordance with claim 1, wherein the respective sensor receptacle comprises an abutment region for the sensor part, the respective through-opening being formed in the abutment region, and wherein the respective sensor receptacle has a depression into which the sensor part is insertable in a positive-locking manner.

3. The adapter device in accordance with claim 1, wherein the through-openings have an extension in the direction of extension of the fluid conduit and/or wherein the fluid conduit receptacle is configured to extend in a straight line.

4. The adapter device in accordance with claim 1, wherein the fluid conduit receptacle comprises at least one groove into which the fluid conduit is insertable at least in part.

5. The adapter device in accordance with claim 4, wherein two complementary grooves are provided which are opposite to one another transversely to the direction of extension and in which the fluid conduit engages in each case in part.

6. The adapter device in accordance with claim 1, wherein the fluid conduit receptacle is formed by the adapter parts, wherein at least one groove extending in the direction of extension for the fluid conduit is arranged or formed on the first adapter part and/or on the second adapter part.

7. The adapter device in accordance with claim 1, wherein the adapter device comprises at least one intermediate part which is formed separate from the adapter parts and is arranged between the adapter parts and comprises or forms the fluid conduit receptacle.

8. The adapter device in accordance with claim 7, wherein a respective receptacle for the intermediate part in which the at least one intermediate part is received at least in part is formed on both adapter parts, the intermediate part engaging in part in each receptacle.

9. The adapter device in accordance with claim 7, wherein at least one receptacle for at least one of the adapter parts is formed on the at least one intermediate part, which adapter part engages in the receptacle at least in portions.

10. The adapter device in accordance with claim 7, the at least one intermediate part comprising a first intermediate part and at least one second intermediate part, wherein the fluid conduit receptacles of the first intermediate part and the at least one second intermediate part are of different sizes for receiving fluid conduits of different diameters.

11. The adapter device in accordance with claim 1, wherein the adapter device comprises at least one receiving part which comprises or forms at least one adapter part receptacle for at least one of the adapter parts, it being possible for the adapter part to be arranged in the at least one adapter part receptacle.

12. The adapter device in accordance with claim 11, wherein the adapter part is designed to be arranged in the at least one adapter part receptacle in a positive-locking manner.

13. The adapter device in accordance with claim 12, wherein the at least one receiving part comprises two adapter part receptacles, it being possible to arrange each adapter part at least in portions in one of the adapter part receptacles,

14. The adapter device in accordance with claim 13, wherein the adapter part receptacles have an offset relative to one another in the direction of extension.

15. The adapter device in accordance with claim 11, wherein the adapter device comprises at least one intermediate part which is formed separate from the adapter parts and is arranged between the adapter parts and comprises or forms the fluid conduit receptacle, and wherein the at least one intermediate part is, comprises or forms the at least one receiving part.

16. The adapter device in accordance with claim 11, wherein the at least one receiving part comprises a first receiving part and at least one second receiving part, wherein the first receiving part and the at least one second receiving part each comprise at least one adapter part receptacle for one of the adapter parts, wherein an offset in the extension direction of the sensor receptacles of the adapter parts connected to the first receiving part and at least one second receiving part is different from one another and is the greater the larger the diameter of the fluid conduit to be used with the respective adapter device.

17. The adapter device in accordance with claim 1, wherein the adapter device comprises a holding part on which one of the adapter parts is secured or is securable, and wherein the respective other adapter part is transferable from a connected state, in which it is connected directly or indirectly to the first-mentioned adapter part, into a maintenance state, in which the adapter part is detachably securable to the holding part and the fluid conduit receptacle is exposed.

18. An adapter system comprising a plurality of adapter devices in accordance with claim 1 having a respective first adapter part and a respective second adapter part wherein the first adapter parts are interconnected with one another and/or wherein the second adapter parts are interconnected with one another.

19. A measuring device comprising at least one adapter device in accordance with claim 1, a sensor device having a first sensor part and a second sensor part, wherein the first sensor part is connected to the first adapter part and the second sensor part is connected to the second adapter part of the respective adapter device, and at least one fluid conduit which is arranged in the fluid conduit receptacle between the adapter parts, wherein the adapter parts are directly or indirectly interconnected, and the contact elements of the sensor parts directly abut against the at least one fluid conduit without intermediate elements.

20. The measuring device in accordance with claim 19, the first sensor part comprising a sealing element attached to a housing of the first sensor part, and the second sensor part comprising a sealing element attached to a housing of the second sensor part, the respective sealing element comprising a cutout which is aligned with the through-opening, the contact element of the first sensor part and of the second sensor part extending through the cutout of the respective sealing element.
